(19) United States
(12) Patent Application Publication
(10) Pub. No.: US 2024/0391901 A1
(43) Pub. Date: Nov. 28, 2024

(54) SUBSTITUTED HETEROCYCLIC COMPOUND CONTAINING ALPHA-KETONE FRAMEWORK, AND USE THEREOF

(71) Applicant: CHINA PHARMACEUTICAL UNIVERSITY, Nanjing, Jiangsu (CN)

(72) Inventors: Jinxin Wang, Nanjing, Jiangsu (CN); Lili Li, Nanjing, Jiangsu (CN); Tao Meng, Nanjing, Jiangsu (CN); Yanguo Shang, Nanjing, Jiangsu (CN); Qingjing Hao, Nanjing, Jiangsu (CN); Yihe Jia, Nanjing, Jiangsu (CN)

(21) Appl. No.: 18/694,448

(22) PCT Filed: Sep. 23, 2022
(86) PCT No.: PCT/CN2022/120795

§ 371 Date: Mar. 22, 2024

(30) Foreign Application Priority Data
Sep. 24, 2021 (CN) 202111118406.6

(51) Int. Cl. C07D 401/14; A61K 31/454; A61K 31/4545; A61K 31/4709; A61P 25/24; C07D 405/14; C07D 413/14; C07D 417/14

(52) U.S. Cl. CPC C07D 401/14; A61K 31/454; A61K 31/4545; A61K 31/4709; A61P 25/24; C07D 405/14; C07D 413/14; C07D 417/14

(57) ABSTRACT

Disclosed in the present invention are a substituted heterocyclic compound containing an α-ketone framework, and the use thereof, which relate to the field of medicinal chemistry. Specifically disclosed are a substituted heterocyclic compound containing an α-ketone framework, or a pharmaceutically acceptable salt thereof, a pharmaceutical composition containing these compounds, and the medical use thereof, and in particular, the use thereof as a fatty acid amide hydrolase (FAAH) inhibitor in the preparation of a drug for preventing or treating FAAH-related diseases, comprising inflammatory diseases, rheumatoid arthritis, hepatitis, hepatic fibrosis, autoimmune disease, pain, depression, pain and depression comorbidity, autism, social anxiety disorder, Tourette's syndrome, neurode-generative diseases, anxiety and post-traumatic stress disorder (PTSD), cannabinoid use disorders, drug withdrawal and anti-tumor treatment.

### TECHNICAL FIELD

The present invention pertains to the field of medicinal chemistry and relates to a substituted heterocyclic compound containing an α-ketone skeleton. Specifically, it involves a substituted heterocyclic FAAH inhibitor containing an α-ketone skeleton, and its application in the preparation of drugs for preventing and/or treating a FAAH-related disease.

### BACKGROUND

Chronic pain is not an early warning of physical injury or illness but rather a result of abnormal neural mechanisms, typically persisting or recurring for more than 3 to 6 months. Increasing evidence suggests that chronic pain is a multifactorial condition that can exacerbate underlying emotional disorders such as anxiety and depression, thereby complicating pharmacological treatment. With a high prevalence and long duration, chronic pain significantly reduces individual health status and quality of life and imposes a considerable economic burden on society as a whole.

Currently, drugs used to treat chronic pain mainly include non-opioid analgesics such as acetaminophen and nonsteroidal anti-inflammatory drugs; opioid drugs such as codeine, hydrocodone, fentanyl, and oxycodone; and when opioids are ineffective, antidepressants such as tricyclic antidepressants and serotonin and norepinephrine reuptake inhibitors, as well as antiepileptic drugs such as gabapentin and pregabalin, which exhibit specific analgesic effects. However, long-term use of nonsteroidal anti-inflammatory drugs can lead to serious gastrointestinal and renal side effects, as well as potentially increased cardiovascular risk. The use of opioid drugs can cause side effects such as respiratory depression, addiction, nausea, confusion, and immunosuppression, making them unsuitable for long-term treatment of chronic pain. Therefore, the development of new analgesic drugs holds significant research value and scientific significance.

The endocannabinoid system is widely distributed throughout the central and peripheral nervous systems, playing a crucial role in regulating the occurrence and development of various diseases such as pain, anxiety, depression, and cardiovascular conditions. It mainly consists of endogenous cannabinoids N-arachidonoylethanolamine (AEA), 2-arachidonoylglycerol (2-AG), cannabinoid receptors (CB1, CB2), and enzymes involved in the synthesis and degradation of endocannabinoids. AEA is the most extensively studied endocannabinoid receptor (CB1, CB2) agonist, which can be hydrolyzed by fatty acid amide hydrolase (FAAH) into ethanolamine and arachidonic acid, thereby becoming inactive within the body.

Research indicates that the protective effects of the endocannabinoid system (ECS) are achieved through the activation of CB receptors. Numerous preclinical studies have demonstrated that gene knockout or pharmacological inhibition leading to FAAH inactivation can increase endogenous AEA levels within the body, subsequently activating CB, TRPV1, and other receptors, thereby exerting various physiological effects such as anti-inflammatory, analgesic, antidepressant, regulation of endocannabinoid dysregulation, and drug withdrawal. The ECS is upregulated in many pathological states such as inflammation, neurodegenerative diseases, gastrointestinal reactions, metabolic and cardiovascular diseases, pain, and cancer, exerting a self-protective role in inhibiting disease progression or reducing signs and symptoms. Therefore, inhibiting the degradation of endogenous cannabinoids represents a therapeutic approach for treating various diseases.

In the organism, endocannabinoids are released into the extracellular space and rapidly cleared through cellular uptake or hydrolysis. Fatty acid amide hydrolase (FAAH) is the primary metabolic enzyme for AEA, regulating the metabolism of endogenous lipid molecules, specifically fatty acid amides. Inhibiting FAAH can elevate endogenous fatty acid amide levels. Studies have shown that FAAH inhibitors can exert antidepressant effects through various pathways, including anti-inflammatory actions, neuroprotection, and improvement of HPA axis dysregulation. Additionally, FAAH inhibitors can induce analgesic effects by activating G proteins, inhibiting adenylate cyclase, and modulating Na⁺/K⁺ ion channels. It has also been discovered that the endocannabinoid system primarily exerts analgesic effects in chronic pain by inhibiting glial cell activation and central sensitization.

By inhibiting FAAH activity, the metabolism of AEA can be reduced, thereby activating CB receptors and exerting analgesic effects. In recent years, the indirect activation of CB receptors through FAAH inhibition has become a research hotspot in the development of analgesic drugs. Some FAAH inhibitors have entered clinical research, providing a new avenue for the development of novel, safe, and effective analgesic drugs, as well as other therapeutics for related diseases.

### CONTENT OF THE PRESENT INVENTION

Objective: The present disclosure provides a substituted heterocyclic compound containing an α-ketone skeleton with FAAH inhibitory activity and use thereof.

Technical solution: To solve the above technical problems, the technical solution of the present disclosure is:

In the first aspect of the present disclosure provides a substituted heterocyclic compound containing an α-ketone skeleton, which is a compound of formula I or a pharmaceutically acceptable salt thereof:

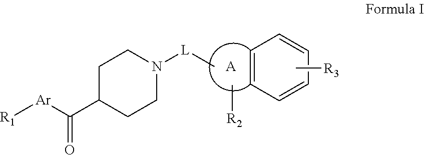

wherein,

Ar is unsubstituted, monosubstituted, or polysubstituted heteroaromatic ring;

R₁ represents H or aryl or heteroaryl;

L represents a linker, selected from —O—, —S—, —NR₄—,

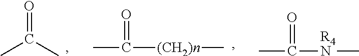

or —(CH₂)ₙ—, n=0, 1, 2, 3;

R₄ is selected from H, C₁-C₈ straight or branched halogenated alkyl, halogenated cycloalkyl, C₁-C₈ straight or branched alkyl, cycloalkyl;

A is a 4-7 membered saturated or unsaturated heterocycle with 1-2 heteroatoms selected from N, S, or O;

R₂, R₃ independently represents H or substituents.

(1) Ar is an unsubstituted, monosubstituted, or polysubstituted heteroaromatic ring, wherein the heteroaromatic ring is selected from: furan, thiophene, pyrrole, oxazole, isoxazole, imidazole, pyrazole, thiazole, isothiazole, triazole, 1,3,4-oxadiazole, 1,3,4-thiadiazole, 1,2,4-oxadiazole, 1,2,4-thiadiazole, pyridine, pyrimidine, pyridazine, benzoxazole, benzothiazole, purine, quinoline, isoquinoline, indole, indazole, oxazolo[4,5-b]pyridine, oxazolo[5,4-b]pyridine.

In some embodiments, the heteroaromatic ring is selected from the following groups:

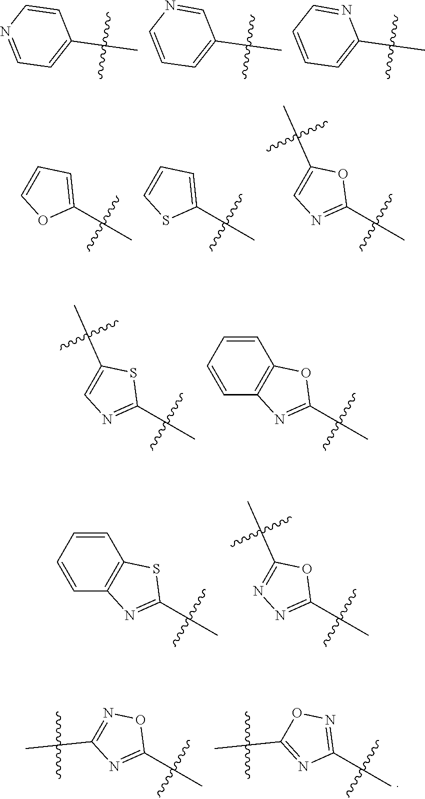

(2) R₁ represents H, aryl or heteroaryl; wherein the aryl or the heteroaryl is unsubstituted, monosubstituted, or polysubstituted, the aryl or the heteroaryl is selected from phenyl, furanyl, thienyl, pyrrolyl, oxazolyl, isooxazolyl, imidazolyl, pyrazolyl, thiazolyl, isothiazolyl, triazolyl, 1,3,4-oxadiazolyl, 1,3,4-thiadiazolyl, 1,2,4-oxadiazolyl, 1,2,4-thiadiazolyl, pyridinyl, pyrimidinyl, pyridazinyl, benzofuranyl, indolyl, quinolinyl, isoquinolinyl, indazolyl, benzoxazolyl, benzothiazolyl, purinyl, oxazolopyridinyl; the substituent on the aryl or the heteroaryl is selected from H, F, Cl, Br, I, CF₃, CN, hydroxyl, methoxy, methyl, nitro, thiol, carboxyl, vinyl, bromomethyl, cyanomethyl, C₁-C₁₀ straight or branched alkoxy, C₁-C₁₀ straight or branched halogenated alkyl, halogenated cycloalkyl, C₁-C₁₀ straight or branched alkyl, cycloalkyl, C₁-C₁₀ straight or branched alkyl substituted thiol group, C₁-C₁₀ straight or branched alkyl amide group, C₁-C₁₀ straight or branched alkyl ester group.

In some embodiments, in R₁, the aryl is selected from:

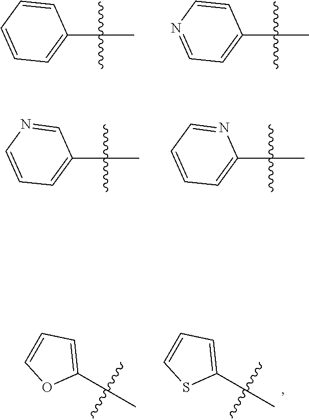 [SMILES: *c1ccccc1 ; *c1ccncc1 ; *c1cccnc1 ; *c1ccccn1 ; *c1ccco1 ; *c1cccs1]

the substituent on aryl is selected from H, F, Cl, Br, I, methoxy, and methyl.

In some embodiments, L is selected from —CO— or —CH₂—;

and/or, A is a 5-6 membered saturated or unsaturated heterocycle with 1-2 heteroatoms selected from N or O;

and/or, R₂ is selected from H, methyl, ethyl, acetyl, phenyl, pyridyl, methoxycarbonyl, propionyl, methoxyacetyl, ethoxyacetyl; preferably H, methyl;

and/or, R₃ is selected from H, F, Cl, Br, I, CF₃, CN, hydroxyl, methoxy, methyl, nitro, thiol, carboxyl, vinyl, bromomethyl, cyanomethyl, aryl, heterocyclyl, C₁-C₁₀ straight or branched alkyl, cycloalkyl, C₁-C₁₀ straight or branched alkoxy, C₁-C₁₀ straight or branched alkyl substituted thiol group, C₁-C₁₀ straight or branched alkyl ester group; preferably H, F, Cl, Br, I.

In some embodiments,

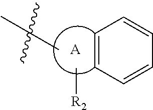

is selected from the following groups:

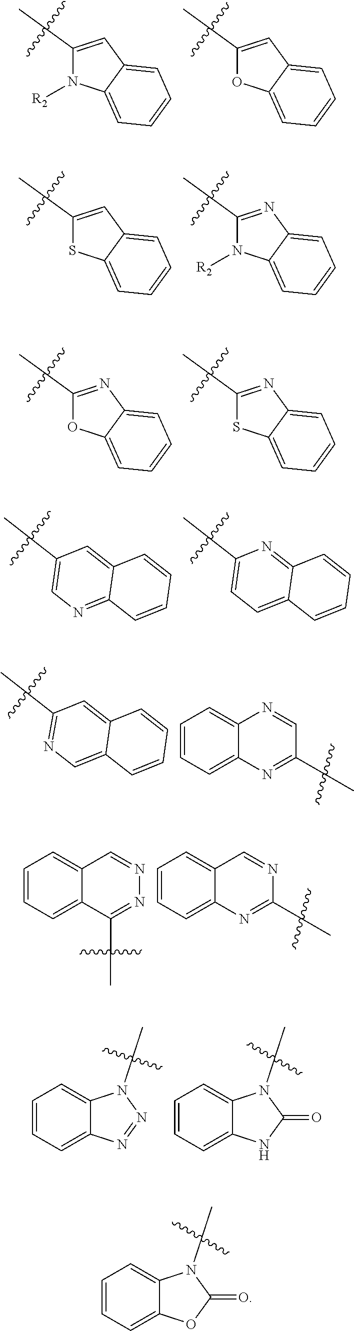

Furthermore, the compound of formula I is selected from:

| Compound | Structure | Name |
|---|---|---|
| I-1 | 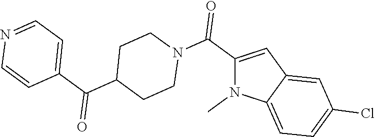 [SMILES: Cn1c(C(=O)N2CCC(C(=O)c3ccncc3)CC2)cc2cc(Cl)ccc21] | (5-Chloro-1-methyl-1H- indol-2-yl)(4- isonicotinamidopiperidin- 1-yl)methanone |
| I-2 | 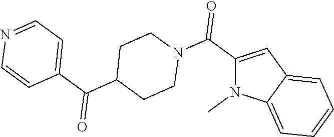 [SMILES: Cn1c(C(=O)N2CCC(C(=O)c3ccncc3)CC2)cc2ccccc21] | (4- Isonicotinamidopiperidin- 1-yl)(1-methyl-1H-indol- 2-yl)methanone |
| I-3 | 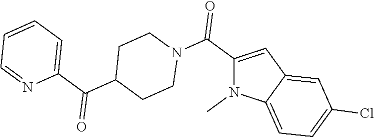 [SMILES: Cn1c(C(=O)N2CCC(C(=O)c3ccccn3)CC2)cc2cc(Cl)ccc21] | (5-Chloro-1-methyl-1H- indol-2-yl)(4-piperidin-1- ylpiperidin-1- yl)methanone |
| I-4 | 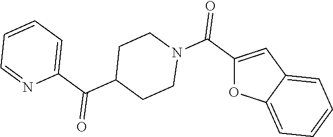 [SMILES: O=C(c1ccccn1)C1CCN(C(=O)c2cc3ccccc3o2)CC1] | (1-(benzofuran-2- carbonyl)piperidin-4- yl)(pyridin-2- yl)methanone |
| I-5 | 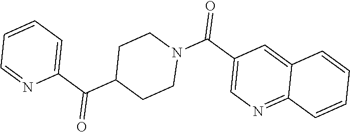 [SMILES: O=C(c1ccccn1)C1CCN(C(=O)c2cnc3ccccc3c2)CC1] | (4-piperidin-1- ylpiperidin-1- yl)(quinolin-3- yl)methanone |
| I-6 | 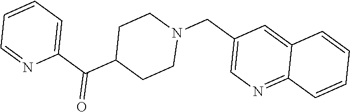 [SMILES: O=C(c1ccccn1)C1CCN(Cc2cnc3ccccc3c2)CC1] | Pyridin-2-yl(1-(quinolin- 3-ylmethyl)piperidin-4- yl)methanone |
| I-7 | 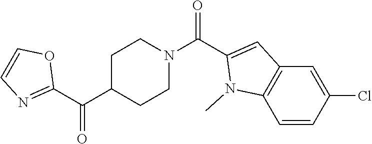 [SMILES: Cn1c(C(=O)N2CCC(C(=O)c3ncco3)CC2)cc2cc(Cl)ccc21] | (5-Chloro-1-methyl-1H- indol-2-yl)(4-(oxazole-2- carbonyl)piperidin-1-yl) methanone |
| I-8 | 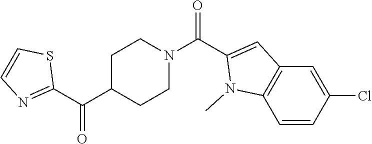 [SMILES: Cn1c(C(=O)N2CCC(C(=O)c3nccs3)CC2)cc2cc(Cl)ccc21] | (5-chloro-1-methyl-1H- indol-2-yl)(4-(thiazol-2- carbonyl)piperidin-1- yl)methanone |
| I-9 | 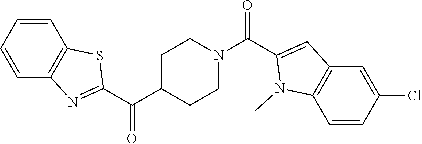 [SMILES: Cn1c(C(=O)N2CCC(C(=O)c3nc4ccccc4s3)CC2)cc2cc(Cl)ccc21] | Benzothiazole-2-yl(1-(5- chloro-1-methyl-1H- indol-2-yl)piperidin-4- yl)methanone |
| I-10 | 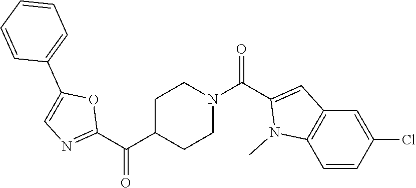 [SMILES: Cn1c(C(=O)N2CCC(C(=O)c3ncc(-c4ccccc4)o3)CC2)cc2cc(Cl)ccc21] | (5-chloro-1-methyl-1H- indol-2-yl)-(4-(5-phenyl- oxazol-2- carbonyl)piperidin-1- yl)methanone |
| I-11 | 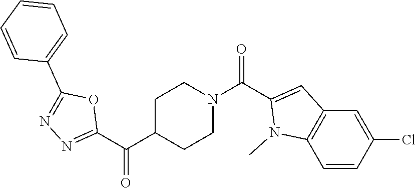 [SMILES: Cn1c(C(=O)N2CCC(C(=O)c3nnc(-c4ccccc4)o3)CC2)cc2cc(Cl)ccc21] | (5-Chloro-1-methyl-1H- indol-2-yl)(4-(5-phenyl- 1,3,4-thiadiazol-2- carbonyl)piperidin-1- yl)methanone |
| I-12 | 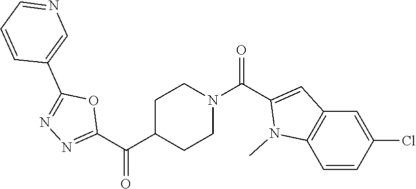 [SMILES: Cn1c(C(=O)N2CCC(C(=O)c3nnc(-c4cccnc4)o3)CC2)cc2cc(Cl)ccc21] | (5-Chloro-1-methyl-1H- indol-2-yl)(4-(5-(pyridin- 3-yl)-1,3,4-thiadiazol-2- carbonyl)piperidin-1- yl)methanone |
| I-13 | 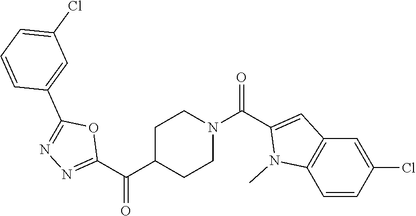 [SMILES: Cn1c(C(=O)N2CCC(C(=O)c3nnc(-c4cccc(Cl)c4)o3)CC2)cc2cc(Cl)ccc21] | (5-chloro-1-methyl-1H- indol-2-yl)(4-(5-(3- chlorophenyl)-1,3,4- oxadiazol-2- carbonyl)piperidin-1- yl)methanone |
| I-14 | 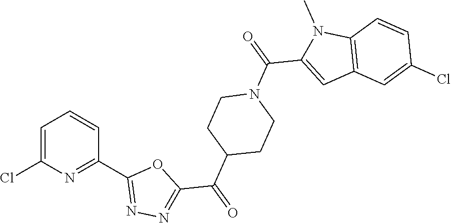 [SMILES: Cn1c(C(=O)N2CCC(C(=O)c3nnc(-c4cccc(Cl)n4)o3)CC2)cc2cc(Cl)ccc21] | (5-chloro-1-methyl-1H- indol-2-yl)(4-(5-(3- chloropyridin-2-yl)-1,3,4- oxadiazol-2- carbonyl)piperidin-1- yl)methanone |
| I-15 | 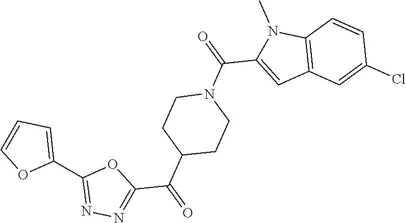 [SMILES: Cn1c(C(=O)N2CCC(C(=O)c3nnc(-c4ccco4)o3)CC2)cc2cc(Cl)ccc21] | (5-chloro-1-methyl-1H- indol-2-yl)(4-(5-(furan-2- yl)-1,3,4-oxadiazol-2- carbonyl)piperidin-1- yl)methanone |
| I-16 | 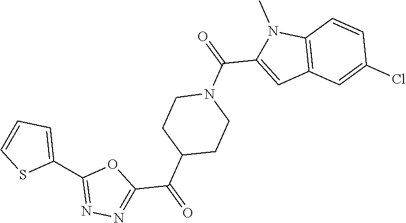 [SMILES: Cn1c(C(=O)N2CCC(C(=O)c3nnc(-c4cccs4)o3)CC2)cc2cc(Cl)ccc21] | (5-chloro-1-methyl-1H- indol-2-yl) (4-(5- (thiophene-2-yl)-1,3,4- oxadiazol-2- carbonyl)piperidin-1- yl)methanone |
| I-17 | 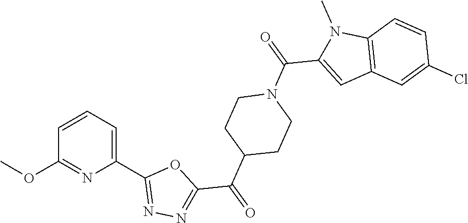 [SMILES: COc1cccc(-c2nnc(C(=O)C3CCN(C(=O)c4cc5cc(Cl)ccc5n4C)CC3)o2)n1] | (5-Chloro-1-methyl-1H- indol-2-yl)(4-(5-(3- methoxypyridin-2-yl)- 1,3,4-oxadiazol-2- yl)carbonyl)piperidin-1- yl)methanone |
| I-18 | 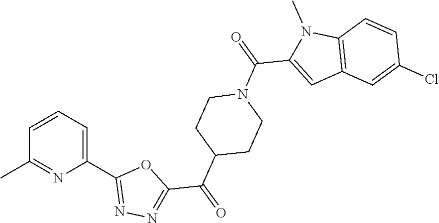 [SMILES: Cc1cccc(-c2nnc(C(=O)C3CCN(C(=O)c4cc5cc(Cl)ccc5n4C)CC3)o2)n1] | (5-Chloro-1-methyl-1H- indol-2-yl)(4-(5-(3- methylpyridin-2-yl)- 1,3,4-oxadiazol-2- yl)piperidin-1- yl)methanone |

In the second aspect of the present disclosure provides a preparation method for the compound of formula I,

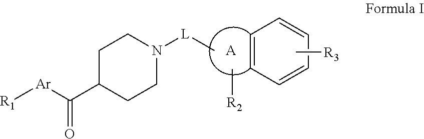

When R₁ is H, Ar is furan, thiophene, pyridine, or fused-heterocycle such as benzofuranyl, the first preparation method is used to prepare Intermediate 1:

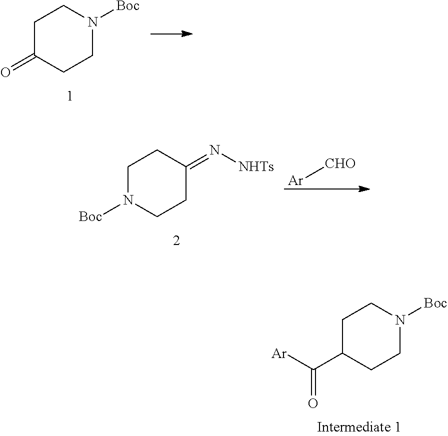

When R₁ is H and Ar is a single heterocycle containing two heteroatoms such as oxazole and thiazole, or a fused heterocycle such as benzoxazole and benzothiazole, the second preparation method is used to prepare Intermediate 1:

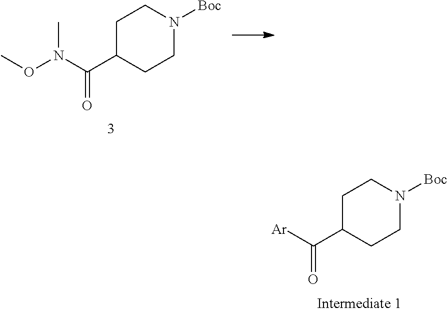

When R₁ is unsubstituted, monosubstituted, or polysubstituted aromatic ring, Ar is furan, 1,3,4-oxadiazole, etc., the third preparation method is used to prepare Intermediate 1:

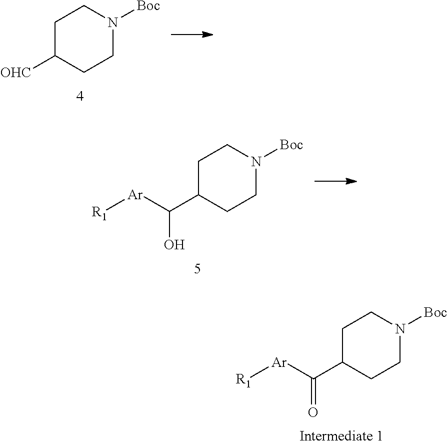

On the other aspect provides a pharmaceutical composition, comprising a therapeutically effective amount of one or more of the compounds, or the pharmaceutically acceptable salt, or the stereoisomer, or the prodrug thereof, and a pharmaceutically acceptable carrier;

the pharmaceutically acceptable carrier is filler, lubricant, emulsifier, wetting agent, colorant, flavoring, stabilizer, antioxidant, preservative.

In the third aspect of the present disclosure provides a use of the compound, the pharmaceutical composition as a FAAH inhibitor, or in the preparation of a drug for preventing and/or treating a FAAH-related disease.

Furthermore, the FAAH-related disease comprises inflammatory disease, rheumatoid arthritis, hepatitis, hepatic fibrosis, autoimmune disease, pain, depression, pain and depression comorbidity, autism, social anxiety disorder, Tourette's syndrome, neurode-generative disease, anxiety and post-traumatic stress disorder (PTSD), cannabinoid use disorder, drug withdrawal and anti-tumor treatment.

The present disclosure proves that a substituted heterocyclic compound containing an α-ketone skeleton has better inhibitory activity against FAAH through large number of modern pharmacological scientific studies, and is suitable for FAAH-related diseases.

### BRIEF DESCRIPTION OF THE DRAWINGS

FIG. 1 is anti-inflammatory activity testing of compounds; (*p<0.01 vs control group, #p<0.05 vs LPS group, ##p<0.01 vs LPS group);

FIG. 2 is sucrose preference experiment study (*p<0.01 vs control group, #p<0.01 vs Model group);

FIG. 3 is open field experiment study (*, P value compared to control group (*P<0.01); ^{#}, P value compared to model group (^{#}P<0.05, ^{##P<}0.01).)

### DETAILED DESCRIPTION OF THE PREFERRED EMBODIMENT

In order to further illustrate the present invention, a series of exemplary embodiments are provided below. These embodiments are purely illustrative and are only intended to provide a detailed description of the present invention, and should not be construed as limiting the scope of the present invention.

### Example 1: Preparation of Compounds

5-Chloro-1-methyl-1H-indole-2-carboxylic acid ethyl ester

Dissolve 5-Chloro-2-indolecarboxylic acid ethyl ester (1 g, 4.47 mmol, 1 eq) in N,N-dimethylformamide, add sodium hydride (215 mg, 8.94 mmol, 2 eq) under nitrogen protection, and stir at 80° C. for 1 hour. Then, add methyl iodide (834 μl, 13.41 mmol, 3 eq) and monitor the reaction progress by TLC. After completion, quench the reaction with saturated ammonium chloride, extract with dichloromethane (3×30 mL), dry the organic phase over anhydrous sodium sulfate, evaporate the solvent under reduced pressure, and purify the crude product by column chromatography to yield a light yellow solid (817 mg) with a yield of 77%. ¹H NMR (400 MHz, DMSO-d₆) δ 7.76 (d, J=2.1 Hz, 1H), 7.64 (d, J=9.0 Hz, 1H), 7.35 (dd, J=8.9, 2.1 Hz, 1H), 7.24 (s, 1H), 4.33 (q, J=7.1 Hz, 2H), 4.02 (s, 3H), 1.34 (t, J=7.1 Hz, 3H) ppm.

5-Chloro-1-methyl-1H-indole-2-carboxylic acid

Dissolve 5-Chloro-1-methyl-1H-indole-2-carboxylic acid ethyl ester (817 mg, 3.45 mmol, 1 eq) in a mixture of methanol and water. Add sodium hydroxide (552 mg, 13.8 mmol, 4 eq) and stir at 60° C. for 2 hours. Then, adjust the pH to 2 with 1 M hydrochloric acid, causing a large amount of solid to precipitate. Filter the mixture, and dry the resulting white solid to obtain 646 mg with a yield of 90%. ¹H NMR (400 MHz, DMSO-d₆) δ 13.10 (s, 1H), 7.74 (d, J=2.1 Hz, 1H), 7.61 (dt, J=9.0, 0.8 Hz, 1H), 7.34-7.31 (m, 1H), 7.19 (d, J=0.9 Hz, 1H) ppm.

1-Methyl-1H-indole-2-carboxylic acid ethyl ester

Using the same synthetic method as for 6-Chloro-1-methyl-1H-indole-2-carboxylic acid ethyl ester, starting with indole-2-carboxylic acid ethyl ester, a light yellow solid was obtained, yielding 235 mg with a yield of 44%. ¹H NMR (400 MHz, CDCl₃-d₆) δ 7.81-7.79 (m, 1H), 7.48-7.45 (m, 2H), 7.38-7.35 (m, 1H), 7.30-7.29 (m, 1H), 4.29 (q, J=6.4 Hz, 2H), 1.36 (t, J=6.4 Hz, 3H) ppm.

1-Methyl-1H-indole-2-carboxylic acid

Dissolve 1-Methyl-1H-indole-2-carboxylic acid ethyl ester (280 mg, 1.38 mmol, 1 eq) in a methanol-water solution, and add sodium hydroxide (221 mg, 5.52 mmol, 4 eq). Stir the reaction mixture at 60° C. for 2 hours with monitoring by TLC. After completion, adjust the pH to 2 with 1 M hydrochloric acid. Only a small amount of solid precipitates, so the organic phase is extracted three times with dichloromethane: methanol=10:1. The combined organic phase is dried over anhydrous sodium sulfate for 30 minutes, filtered, and the filtrate is evaporated under reduced pressure to yield a grey-white solid (203 mg) with a yield of 84%. ¹H NMR (400 MHz, DMSO-d₆) δ 12.96 (s, 1H), 7.64 (dd, J=8.1, 1.1 Hz, 1H), 7.44 (dd, J=8.3, 1.1 Hz, 1H), 7.24 (ddd, J=8.2, 6.9, 1.2 Hz, 1H), 7.09-7.04 (m, 2H), 3.37 (s, 3H) ppm.

4-[(2-Methylbenzenesulfonyl)amino]benzyl]piperidine-1-carboxylate

Dissolve N-Boc-piperidin-4-one (1 g, 5 mmol, 1 eq) and p-toluenesulfonyl hydrazide (935 mg, 5 mmol, 1 eq) in methanol and react at room temperature for 6 hours. After completion, concentrate under reduced pressure to yield a white solid, obtaining 1.56 g with a yield of 84%. ¹H NMR (300 MHz, DMSO-d₆) δ 10.29 (s, 1H), 7.79-7.71 (m, 2H), 7.42 (d, J=8.0 Hz, 2H), 3.41 (q, J=6.3 Hz, 4H), 2.41 (s, 3H), 2.37 (d, J=6.1 Hz, 2H), 2.24 (t, J=6.1 Hz, 2H), 1.42 (s, 9H) ppm.

4-Pyridinylpiperidine-1-carboxylate tert-butyl ester

Dissolve 4-[(2-methylphenyl)sulfonyl]benzyl]piperidine-1-carboxylate tert-butyl ester (500 mg, 1.36 mmol, 1 eq) and 3-pyridinecarboxaldehyde (127 μl, 1.36 mmol, 1 eq) in anhydrous 1,4-dioxane. Add cesium carbonate (220 mg, 2.04 mmol, 1.5 eq), and stir the mixture at 110° C. for 3 hours. After completion, quench the reaction with saturated ammonium chloride solution, then extract with dichloromethane (3×35 mL). Combine the organic phases, dry over anhydrous sodium sulfate, and evaporate the solvent under reduced pressure. Purify the crude product by column chromatography to yield a pale yellow oily substance (190 mg) with a yield of 48%. ¹H NMR (300 MHz, DMSO-d₆) δ 9.19 (dd, J=2.3, 0.9 Hz, 1H), 8.83 (dd, J=4.8, 1.7 Hz, 1H), 8.35 (dt, J=8.0, 2.0 Hz, 1H), 7.61 (ddd, J=7.9, 4.8, 0.9 Hz, 1H), 4.00 (d, J=13.0, 2H), 3.69 (tt, J=11.3, 3.6 Hz, 1H), 2.95 (s, 2H), 1.82 (dd, J=13.6, 3.5 Hz, 2H), 1.50-1.44 (m, 2H), 1.43 (s, 9H) ppm.

4-Isonicotinamidopiperidine-1-carboxylate tert-butyl ester

Dissolve 4-[(2-methylphenyl)sulfonyl]benzyl]piperidine-1-carboxylate tert-butyl ester (500 mg, 1.36 mmol, 1 eq) and 3-pyridinecarboxaldehyde (127 μl, 1.36 mmol, 1 eq) in anhydrous 1,4-dioxane. Add cesium carbonate (220 mg, 2.04 mmol, 1.5 eq), and stir the mixture at 110° C. for 3 hours. After completion, quench the reaction with saturated ammonium chloride solution, then extract with dichloromethane (3×35 mL). Combine the organic phases, dry over anhydrous sodium sulfate, and evaporate the solvent under reduced pressure. Purify the crude product by column chromatography to yield a pale yellow oily substance (388 mg) with a yield of 98%. ¹H NMR (400 MHz, DMSO-d₆) δ 8.83-8.81 (m, 2H), 7.86-7.84 (m, 2H), 3.96 (d, J=13.2 Hz, 2H), 3.63 (tt, J=11.3, 3.6 Hz, 1H), 2.92 (s, 2H), 1.79 (d, J=13.2 Hz, 2H), 1.42-1.39 (m, 11H) ppm.

Piperidin-4-yl(pyridin-4-yl)methanone

Dissolve 4-isonicotinamidopiperidine-1-carboxylate tert-butyl ester in HCl/ethyl acetate, stir at room temperature for half an hour, filter, and dry to obtain a pale yellow solid, yielding 230 mg with a yield of 82%. ¹H NMR (400 MHz, DMSO-d₆) δ 7.82-7.73 (m, 2H), 7.44-7.36 (m, 2H), 3.17-3.14 (m, 4H), 2.76-2.67 (m, 1H), 2.63-2.59 (m, 1H), 2.40-2.37 (m, 4H) ppm.

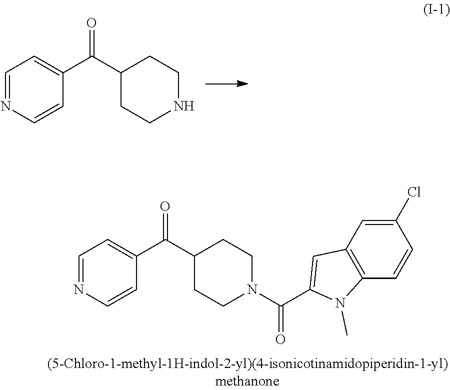 [SMILES: O=C(c1ccncc1)C1CCNCC1 ; Cn1c(C(=O)N2CCC(C(=O)c3ccncc3)CC2)cc2cc(Cl)ccc21]

Dissolve 4-piperidin-4-yl(pyridin-4-yl)methanone (200 mg, 0.885 mmol, 1.0 eq) and 5-chloro-1-methyl-1H-indole-2-carboxylic acid (185 mg, 0.885 mmol, 1.0 eq) in N,N-dimethylformamide. Add EDCI (85 mg, 0.442 mmol, 0.5 eq), 1-hydroxybenzotriazole (60 mg, 0.442 mmol, 0.5 eq), and N-methylmorpholine (195 μl, 1.77 mmol, 1.0 eq). Stir the mixture at room temperature for 0.5 hours, then add 30 mL of water and extract with ethyl acetate (3×30 mL). Combine the organic phases, dry over anhydrous sodium sulfate, and evaporate the solvent under reduced pressure. Purify the crude product by column chromatography to yield a white solid (112 mg) with a yield of 33%. ¹H NMR (400 MHz, DMSO-d6) δ 8.84-8.83 (m, 2H), 7.88-7.87 (m, 2H), 7.65 (d, J=2.1 Hz, 1H), 7.56 (d, J=8.8 Hz, 1H), 7.24 (dd, J=8.8, 2.1 Hz, 1H), 6.65 (s, 1H), 4.51 (s, 1H), 4.09-3.93 (m, 1H), 3.84-3.78 (m, 1H), 3.76 (s, 3H), 3.18-2.17 (m, 2H), 2.02-1.90 (m, 2H), 1.61-1.51 (m, 2H) ppm; HRMS (ESI+): m/z [M+H]+ calcd for C₂₁H₂₁ClN₃O₂, 382.1317; found 382.1370.

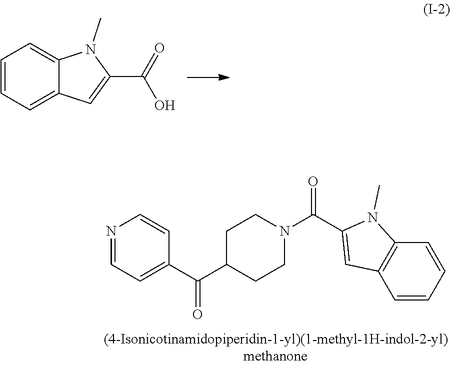 [SMILES: Cn1c(C(=O)O)cc2ccccc21 ; Cn1c(C(=O)N2CCC(C(=O)c3ccncc3)CC2)cc2ccccc21]

Using the same synthetic method as for (5-Chloro-1-methyl-1H-indol-2-yl) (4-isonicotinamidopiperidin-1-yl)methanone, starting with piperidin-4-yl(pyridin-4-yl)methanone and 1-methyl-1H-indole-2-carboxylic acid, a beige solid (208 mg) with a yield of 68%. ¹H NMR (400 MHz, DMSO-d₆) δ 8.84-8.82 (m, 2H), 7.89-7.87 (m, 2H), 7.61-7.59 (m, 1H), 7.51 (d, J=8.3 Hz, 1H), 7.25 (tt, J=8.0, 1.1 Hz, 1H), 7.12-7.07 (m, 1H), 6.66 (s, 1H), 4.46-4.10 (m, 2H), 3.83-3.78 (m, 1H), 3.76 (s, 3H), 3.23-3.18 (m, 2H), 1.90 (s, 2H), 1.61-1.51 (m, 2H) ppm; ¹³C NMR (101 MHz, CDCl₃) δ 200.95, 162.73, 151.19, 141.63, 136.28, 132.86, 127.28, 125.97, 123.75, 121.19, 120.75, 110.95, 102.70, 43.62, 31.32, 28.37 ppm; HRMS (ESI⁺): m/z [M+H]⁺ calcd for C₂₁H₂₁N₃O₂, 348.1707; found 348.1757.

4-Pyridinecarboxylic acid tert-butyl ester

Using the same synthetic method as for 4-isonicotinamidopiperidine-1-carboxylate tert-butyl ester, starting with 4-[(2-methylphenyl)sulfonyl]benzyl]piperidine-1-carboxylate tert-butyl ester and 2-pyridinecarboxaldehyde, a pale yellow oil (249 mg) was obtained with a yield of 63%. ¹H NMR (300 MHz, DMSO-d₆) δ 7.74-7.70 (m, 2H), 7.40-7.37 (m, 2H), 3.41-3.37 (m, 4H), 2.38-2.33 (m, 4H), 2.23-2.19 (m, 1H), 1.42 (s, 9H) ppm.

Piperidin-4-yl(pyridin-2-yl)methanone

Dissolve 4-pyridinecarboxylic acid tert-butyl ester in HCl/ethyl acetate, stir at room temperature for half an hour, filter, and dry to obtain a light yellow solid (172 mg) with a yield of 88%. ¹H NMR (400 MHz, DMSO-d₆) δ 8.77-8.75 (m, 1H), 8.08-8.00 (m, 2H), 7.73-7.70 (m, 1H), 4.07 (tt, J=11.4, 3.7 Hz, 1H), 3.29 (dt, J=12.8, 3.3 Hz, 2H), 3.03 (dtd, J=12.7, 10.0, 2.8 Hz, 2H), 2.00 (dd, J=14.1, 3.6 Hz, 2H), 1.87-1.76 (m, 2H) ppm.

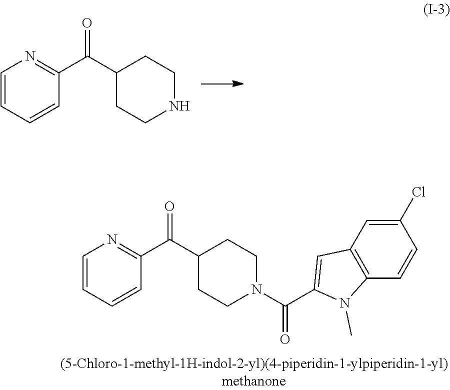 [SMILES: O=C(c1ccccn1)C1CCNCC1 ; Cn1c(C(=O)N2CCC(C(=O)c3ccccn3)CC2)cc2cc(Cl)ccc21]

Using the same synthetic method as for (5-chloro-1-methyl-1H-indol-2-yl) (4-isonicotinamidopiperidin-1-yl)methanone, starting with piperidin-4-yl(pyridin-2-yl)methanone and 5-chloro-1-methyl-1H-indole-2-carboxylic acid, a light yellow solid (53 mg) was obtained with a yield of 29%. ¹H NMR (400 MHz, DMSO-d₆) δ 8.77 (ddd, J=4.7, 1.7, 1.0 Hz, 1H), 8.06-7.96 (m, 2H), 7.69 (ddd, J=7.3, 4.7, 1.5 Hz, 1H), 7.66 (d, J=2.0 Hz, 1H), 7.56 (d, J=8.8 Hz, 1H), 7.25 (dd, J=8.8, 2.1 Hz, 1H), 6.65 (d, J=0.8 Hz, 1H), 4.55 (s, 1H), 4.20-4.12 (m, 1H), 4.03-3.93 (m, 1H), 3.76 (s, 3H), 3.31-3.06 (m, 2H), 1.95 (s, 2H), 1.63-1.53 (m, 2H) ppm; HRMS (ESI⁺): m/z [M+H]⁺ calcd for C₂₁H₂₁ClN₃O₂, 382.1317; found 382.1365.

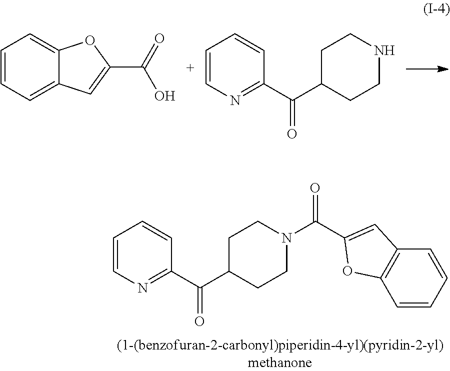

Using the same synthetic method as for (5-chloro-1-methyl-1H-indol-2-yl) (4-isonicotinamidopiperidin-1-yl)methanone, starting with benzofuran-2-carboxylic acid and piperidin-4-yl(pyridin-2-yl)methanone, a white solid (137 mg) was obtained with a yield of 46.44%. ¹H NMR (500 MHz, Chloroform-d) δ 8.64 (dd, J=4.2, 1.7 Hz, 1H), 7.87 (dd, J=7.8, 1.3 Hz, 1H), 7.79-7.65 (m, 3H), 7.56 (d, J=1.9 Hz, 1H), 7.46-7.37 (m, 2H), 7.32 (ddd, J=7.3, 4.2, 1.3 Hz, 1H), 3.57 (dd, J=8.5, 5.8 Hz, 2H), 3.49 (dd, J=8.5, 5.8 Hz, 2H), 3.32 (p, J=6.2 Hz, 1H), 2.15 (ddt, J=21.6, 8.6, 6.0 Hz, 4H) ppm.

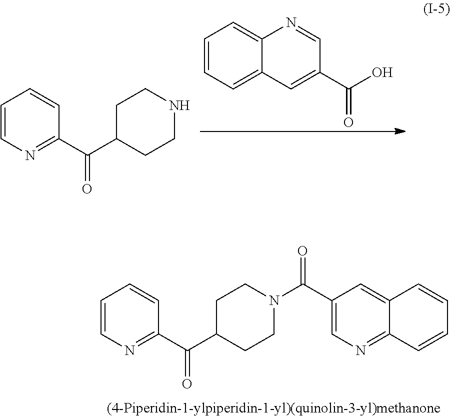

Using the same synthetic method as for (5-Chloro-1-methyl-1H-indol-2-yl) (4-isonicotinamidopiperidin-1-yl)methanone, starting with quinolin-3-carboxylic acid and piperidin-4-yl(pyridin-2-yl)methanone, a white solid (159 mg) was obtained with a yield of 52.18%. ¹H NMR (500 MHz, Chloroform-d) δ 8.84 (d, J=1.6 Hz, 1H), 8.64 (dd, J=4.2, 1.7 Hz, 1H), 8.59 (t, J=1.7 Hz, 1H), 8.01 (ddd, J=8.3, 2.3, 1.0 Hz, 1H), 7.88 (ddd, J=12.4, 7.5, 1.2 Hz, 2H), 7.72 (td, J=7.6, 1.6 Hz, 1H), 7.65 (td, J=7.5, 1.1 Hz, 1H), 7.49 (td, J=8.3, 1.4 Hz, 1H), 7.32 (ddd, J=7.3, 4.2, 1.3 Hz, 1H), 3.64 (dd, J=8.5, 5.8 Hz, 2H), 3.59 (dd, J=8.5, 5.8 Hz, 2H), 3.34 (p, J=6.2 Hz, 1H), 2.17 (dt, J=8.4, 6.0 Hz, 2H), 2.08 (dt, J=8.4, 5.9 Hz, 2H) ppm.

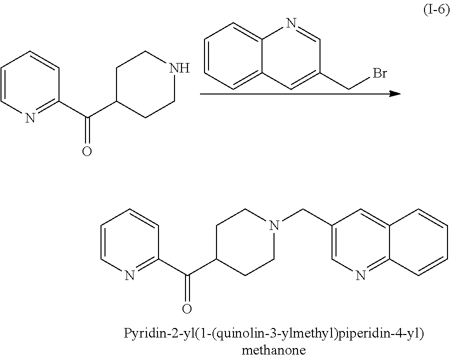

Dissolve piperidin-4-yl(pyridin-2-yl)methanone (200 mg, 1.05 mmol, 1 eq) in acetonitrile. Add potassium carbonate (647 mg, 4.21 mmol, 4 eq) and potassium iodide (388 mg, 2.10 mmol, 2 eq), then stir at room temperature for 30 minutes. Add 3-(bromomethyl)quinoline (218 mg, 1.58 mmol, 1.5 eq) and heat the mixture to 80° C. Stir for 2.5 hours, then filter. The filtrate is extracted with dichloromethane (3×30 mL). The combined organic phases are washed with 10% potassium hydrogen phosphate solution (3×30 mL) and saturated brine (3×30 mL). Then, add anhydrous sodium sulfate to dry, and evaporate the solvent under reduced pressure. Purify the crude product by column chromatography to obtain a light yellow solid (109 mg) with a yield of 31%. ¹H NMR (300 MHz, DMSO-d₆) δ 8.68 (dd, J=4.2, 1.4 Hz, 1H), 8.59 (d, J=1.6 Hz, 1H), 8.04-7.95 (m, 3H), 7.93-7.79 (m, 2H), 7.85-7.75 (m, 1H), 7.62-7.47 (m, 2H), 7.52-7.40 (m, 1H), 3.71 (s, 1H), 3.22 (q, J=6.1 Hz, 1H), 3.02-2.77 (m, 3H), 2.07-1.80 (m, 4H) ppm.

4-(oxazole-2-carbonyl)piperidine-1-carboxylate tert-butyl ester

Oxazole (284 μl, 4.32 mmol, 1.2 eq) was dissolve in anhydrous THF, and LDA (3.6 ml, 7.2 mmol, 2 eq) was slowly added at −40° C. After stirring for 30 minutes, the solution of Boc-4-[(methoxy)methylamino]piperidine (1 g, 3.6 mmol, 1 eq) in THF was added, and the reaction mixture was allowed to warm to room temperature overnight. After completion of the reaction, quench the reaction mixture with saturated ammonium chloride solution, followed by extraction with EA three times. Combine the organic phases, wash them three times with saturated brine, dry over anhydrous sodium sulfate, and then purify by column chromatography to obtain 228 mg of the product with a yield of 22%. ¹H NMR (400 MHz, CDCl₃-d) δ 8.19 (d, J=0.8 Hz, 1H), 7.62 (d, J=0.9 Hz, 1H), 3.84-3.79 (m, 2H), 3.75-3.69 (m, 2H), 3.24-3.19 (m, 1H), 2.14-2.04 (m, 4H), 1.45 (s, 9H) ppm.

Oxazole-2-yl(piperidin-4-yl)methanone

The 4-(oxazole-2-carbonyl)piperidine-1-carboxylate tert-butyl ester was dissolved in a small amount of ethyl acetate, followed by addition of HCl/ethyl acetate. The reaction proceeded at room temperature for 30 minutes, resulting in a significant amount of white solid precipitation. After filtration and drying, a white solid weighing 134 mg was obtained with a yield of 87%. ¹H NMR (400 MHz, DMSO-d₆) δ 9.10 (s, 1H), 8.74 (s, 1H), 3.29-3.23 (m, 2H), 3.11 (s, 2H), 2.99-2.89 (m, 2H), 1.85-1.72 (m, 4H) ppm.

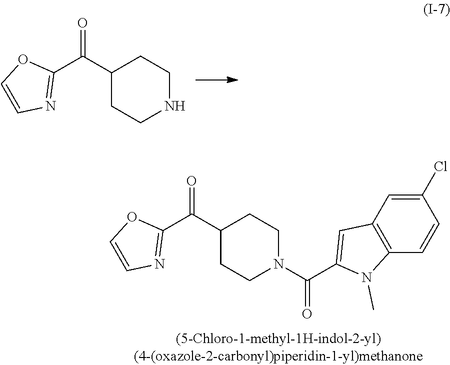 [SMILES: O=C(c1ncco1)C1CCNCC1 ; Cn1c(C(=O)N2CCC(C(=O)c3ncco3)CC2)cc2cc(Cl)ccc21]

Using the synthesis method described for (5-chloro-1-methyl-1H-indol-2-yl) (4-isothiocyanatopiperidin-1-yl)methanone, 79 mg of solid was obtained with a yield of 23%. ¹H NMR (400 MHz, DMSO-d₆) δ 8.44 ((d, J=1.7 Hz, 1H), 7.67-7.65 (m, 1H), 7.59-7.55 (m, 2H), 7.27-7.23 (m, 1H), 6.65 (s, 1H), 4.52 (s, 1H), 4.04-3.93 (m, 1H), 3.76 (s, 3H), 3.73-3.69 (m, 1H), 3.17-3.11 (m, 2H), 2.01-2.00 (m, 2H), 1.66-1.56 (m, 2H) ppm; HRMS (ESI⁻): m/z [M+H]⁺ calcd for C₁₉H₁₉ClN₃O₃, 372.1109; found 372.1164.

4-(thiazole-2-carbonyl)piperidine-1-carboxylate tert-butyl ester

Thiazole (312 μl, 4.41 mmol, 1.2 eq) was dissolved in anhydrous THF, and LDA (3.6 ml, 7.2 mmol, 2 eq) was slowly added at −40° C. After stirring for 30 minutes, a THF solution of 1-Boc-4-[(methoxy)methylamino]benzylamine (1 g, 3.67 mmol, 1 eq) was added, and the reaction mixture was allowed to warm to room temperature overnight. Upon completion of the reaction, the mixture was quenched with saturated ammonium chloride solution, and then extracted three times with EA. The combined organic phases were washed three times with saturated saline solution, dried over anhydrous sodium sulfate, and finally purified by column chromatography to afford 228 mg of a light yellow oily substance, with a yield of 22%. ¹H NMR (400 MHz, CDCl₃-d) δ 7.90 (d, J=4.8 Hz, 1H), 7.62 (d, J=4.6 Hz, 1H), 3.79-3.74 (m, 2H), 3.48-3.43 (m, 2H), 3.27-3.23 (m, 1H), 2.24-2.11 (m, 4H), 1.45 (s, 9H) ppm.

Piperidine-4-yl(thiazol-2-yl)methanone

4-(Thiazol-2-carbonyl)piperidine-1-carboxylate tert-butyl ester was dissolved in a small amount of ethyl acetate, and then HCl/ethyl acetate was added. The reaction was carried out at room temperature for 30 minutes, resulting in the precipitation of a large amount of white solid. After filtration and drying, a white solid weighing 376 mg was obtained with a yield of 87%. ¹H NMR (400 MHz, DMSO-d₆) δ 9.25 (s, 1H), 8.91 (s, 1H), 3.40 (s, 1H), 3.28-3.22 (m, 2H), 3.01-2.88 (m, 3H), 1.85-1.70 (m, 4H) ppm.

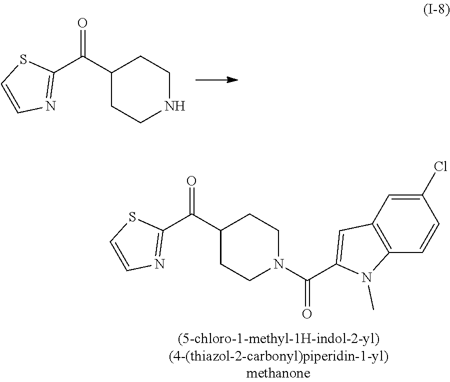 [SMILES: O=C(c1nccs1)C1CCNCC1 ; Cn1c(C(=O)N2CCC(C(=O)c3nccs3)CC2)cc2cc(Cl)ccc21]

According to the synthesis method of (5-chloro-1-methyl-1H-indol-2-yl) (4-(isothiocyanate)piperidin-1-yl)methanone, 237 mg of solid was obtained, with a yield of 47%. ¹H NMR (400 MHz, DMSO-d₆) δ 8.25 (d, J=3.0 Hz, 1H), 8.19 (d, J=3.0 Hz, 1H), 7.66 (d, J=2.0 Hz, 1H), 7.56 (d, J=8.8 Hz, 1H), 7.25 (dd, J=8.8, 2.0 Hz, 1H), 6.65 (d, J=3.0 Hz, 1H), 4.71-4.35 (m, 1H), 4.21-3.96 (m, 1H), 3.95-3.83 (m, 1H), 3.76 (s, 3H), 3.31-3.00 (m, 2H), 2.16-1.90 (m, 2H), 1.78-1.54 (m, 2H) ppm.

4-((1-benzothiophen-2-yl)carbonyl)piperidine-1-carboxylate tert-butyl ester

Using the same synthetic method as for 4-(thiazole-2-carbonyl)piperidine-1-carboxylate tert-butyl ester, starting with benzothiazole, resulting in a light yellow solid of 325 mg with a yield of 51%. ¹H NMR (300 MHz, DMSO-d₆) δ 8.33-8.25 (m, 2H), 7.73-7.64 (m, 2H), 4.05 (d, J=13.2 Hz, 2H), 3.90 (tt, J=11.4, 3.6 Hz, 1H), 2.97 (s, 2H), 2.04-1.99 (m, 2H), 1.65-1.45 (m, 2H), 1.44 (s, 9H) ppm.

Benzothiazole-2-carbaldehyde (4-piperidinyl)

Benzothiazole-2-carbaldehyde (4-piperidinyl)methanone was dissolved in a small amount of ethyl acetate (EA), and then HCl/EA was added. The mixture was allowed to react at room temperature for 30 minutes, resulting in the precipitation of a large amount of white solid. After filtration and drying, a white solid weighing 256 mg was obtained, with a yield of 97%. ¹H NMR (300 MHz, DMSO-d₆) δ 8.33-8.23 (m, 2H), 7.74-7.65 (m, 2H), 4.02 (tt, J=11.2, 3.7 Hz, 1H), 3.35 (s, 2H), 3.18-3.06 (m, 2H), 2.22-2.17 (m, 2H), 2.01-1.87 (m, 2H) ppm.

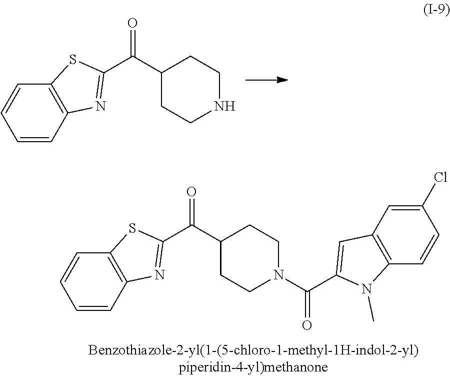 [SMILES: O=C(c1nc2ccccc2s1)C1CCNCC1 ; Cn1c(C(=O)N2CCC(C(=O)c3nc4ccccc4s3)CC2)cc2cc(Cl)ccc21]

According to the synthesis method described for (5-chloro-1-methyl-1H-indol-2-yl) (4-isocyanophenyl)piperidin-1-yl)methanone, 24 mg of solid was obtained with a yield of 30%. ¹H NMR (300 MHz, DMSO-d₆) δ 8.32-8.27 (m, 2H), 7.76-7.57 (m, 4H), 7.29-7.25 (m, 1H), 6.69 (s, 1H), 4.59 (s, 1H), 4.08-4.06 (m, 2H), 3.79 (s, 3H), 3.42-3.39 (m, 2H), 2.15 (s, 2H), 1.76-1.69 (m, 2H) ppm; HRMS (ESI⁺): m/z [M+H]⁺ calcd for C₂₃H₂₁ClN₃O₂S, 438.1038; found 438.1118.

5-Phenylthiazole

Benzaldehyde (1 g, 9.42 mmol, 1 eq) was dissolved in methanol, followed by the addition of p-toluenesulfonyl isocyanide (2.023 g, 10.362 mmol, 1.1 eq) and K₂CO₃ (2.598 g, 18.8 mmol, 2 eq). The mixture was refluxed at 80° C. for 1.5 hours. After completion of the reaction, methanol was removed by vacuum concentration. The residue was dissolved in dichloromethane (DCM) and filtered to remove insoluble K₂CO₃. The filtrate was concentrated under reduced pressure and purified by column chromatography to yield 1.03 g of solid, with a yield of 74%. ¹H NMR (300 MHz, DMSO-d₆) δ 8.44 (s, 1H), 7.74-7.71 (m, 2H), 7.69 (s, 1H), 7.50-7.45 (m, 2H), 7.40-7.34 (m, 1H) ppm.

4-(5-Phenyl-oxazol-2-ylcarbonyl)piperidine-1-carboxylate tert-butyl ester

5-Phenyl-oxazol (320 mg, 2.208 mmol, 1.2 eq) was dissolved in anhydrous THF, and LDA (1.1 ml, 2.208 mmol, 1.2 eq) was slowly added at −40° C. After stirring for 30 minutes, a THF solution of 1-Boc-4-[(methoxy) methylaminoformyl]piperidine (500 mg, 1.84 mmol, 1 eq) was added, and the mixture was stirred overnight at room temperature. Upon completion of the reaction, it was quenched with saturated ammonium chloride solution, and then extracted three times with EA. The combined organic phases were washed three times with saturated saline solution, dried over anhydrous sodium sulfate, and finally purified by column chromatography to yield 325 mg of solid, with a yield of 37%. ¹H NMR (400 MHz, DMSO-d₆) δ 8.05 (s, 1H), 7.89-7.82 (m, 2H), 7.59-7.51 (m, 2H), 7.51-7.44 (m, 1H), 4.00 (d, J=13.0 Hz, 2H), 3.59 (tt, J=11.5, 3.6 Hz, 1H), 2.90 (s, 2H), 1.92 (dd, J=13.7, 3.5 Hz, 2H), 1.54-1.43 (m, 2H), 1.43-1.39 (m, 9H) ppm.

(5-Phenyl-oxazol-2-yl)-(piperidin-4-yl)methanone

4-(5-Phenyl-oxazol-2-carbonyl)piperidine-1-carboxylate tert-butyl ester was dissolved in a solution of hydrogen chloride in dioxane, and the reaction was carried out at room temperature for 30 minutes. A large amount of white solid precipitated after the reaction. The solid was filtered and dried to yield 152 mg of the product, with a yield of 77%. ¹H NMR (400 MHz, DMSO-d₆) δ 8.08 (s, 1H), 7.90-7.83 (m, 2H), 7.59-7.45 (m, 3H), 3.72 (tt, J=11.3, 3.6 Hz, 1H), 3.33 (d, J=12.8 Hz, 2H), 3.05 (q, J=12.3, 11.9 Hz, 2H), 2.11 (dd, J=14.4, 3.6 Hz, 2H), 1.87 (q, J=11.1 Hz, 2H) ppm.

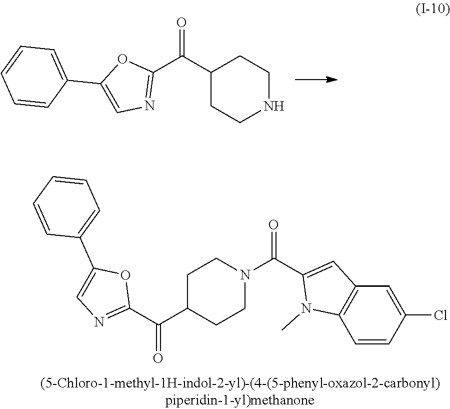 [SMILES: O=C(c1ncc(-c2ccccc2)o1)C1CCNCC1 ; Cn1c(C(=O)N2CCC(C(=O)c3ncc(-c4ccccc4)o3)CC2)cc2cc(Cl)ccc21]

According to the synthesis method of (5-chloro-1-methyl-1H-indol-2-yl)-(4-(isothiocyanato)piperidin-1-yl)methanone, 18 mg of solid was obtained, with a yield of 24%. ¹H NMR (400 MHz, DMSO-d₆) δ 8.08 (d, J=4.1 Hz, 1H), 7.91-7.83 (m, 2H), 7.69-7.66 (m, 1H), 7.60-7.52 (m, 3H), 7.52-7.49 (m, 2H), 7.31-7.19 (m, 1H), 6.67 (s, 1H), 4.55 (s, 1H), 4.05 (s, 1H), 3.77 (s, 4H), 3.71 (s, 1H), 3.25-3.04 (m, 2H), 2.15-1.92 (m, 2H), 1.70-1.57 (m, 2H) ppm.

2-Phenyl-1,3,4-thiadiazole

Benzoyl hydrazide (1 g, 7.345 mmol, 1 eq) was dissolved in trimethyl orthoformate, and p-toluenesulfonic acid (126 mg, 0.7345 mmol, 0.1 eq) was added. The mixture was refluxed at 120° C. for 1.5 hours. After completion of the reaction, the crude product was purified by column chromatography to yield 1.05 g of solid, with a yield of 98%. ¹H NMR (400 MHz, DMSO-d₆) δ 9.36 (s, 1H), 7.91-7.86 (m, 2H), 7.68-7.53 (m, 3H) ppm.

4-(5-Phenyl-1,3,4-thiadiazol-2-carbonyl)piperidine-1-carboxylate tert-butyl ester

2-Phenyl-1,3,4-thiadiazole (323 mg, 2.21 mmol, 1.2 eq) was dissolved in anhydrous THF, and n-BuLi (0.88 mL, 2.21 mmol, 1.2 eq) was slowly added at −40° C. The mixture was stirred for 30 minutes before adding the THE solution of 1-Boc-4-[(methoxy)methylaminoformyl]piperidine (500 mg, 1.84 mmol, 1 eq). The reaction mixture was then allowed to warm to room temperature and stirred overnight. Upon completion of the reaction, it was quenched with saturated ammonium chloride solution, and then extracted three times with EA. The combined organic phases were washed three times with saturated saline solution, dried over anhydrous sodium sulfate, and finally purified by column chromatography to yield a solid product (227 mg, 34% yield). ¹H NMR (300 MHz, DMSO-d₆) δ 8.18-8.09 (m, 2H), 7.80-7.62 (m, 3H), 4.08-3.98 (m, 2H), 3.64 (tt, J=11.4, 3.6 Hz, 1H), 2.95 (d, J=14.8 Hz, 2H), 2.03 (d, J=13.0 Hz, 2H), 1.54 (tt, J=12.1, 6.1 Hz, 2H), 1.44 (s, 9H) ppm.

(5-Phenyl-1,3,4-thiadiazol-2-yl)(piperidin-4-yl)methanone

227 mg of 4-(5-Phenyl-1,3,4-thiadiazol-2-carbonyl)piperidine-1-carboxylate tert-butyl ester was dissolved in hydrochloric acid/dioxane solution. The reaction was stirred at room temperature for 30 minutes, during which a large amount of white solid precipitated. After filtration and drying, a solid weighing 120 mg was obtained, with a yield of 65%. ¹H NMR (400 MHz, DMSO-d₆) δ 9.41-9.10 (m, 2H), 8.11 (dd, J=5.9, 3.8 Hz, 1H), 7.68 (dtd, J=14.9, 7.3, 2.2 Hz, 2H), 3.71-3.67 (m, 3H), 3.28-3.21 (m, 1H), 3.10 (s, 1H), 2.93 (d, J=11.9 Hz, 1H), 2.17 (dd, J=14.3, 3.7 Hz, 1H), 2.03-1.88 (m, 1H), 1.79 (td, J=12.1, 11.3, 5.9 Hz, 2H) ppm.

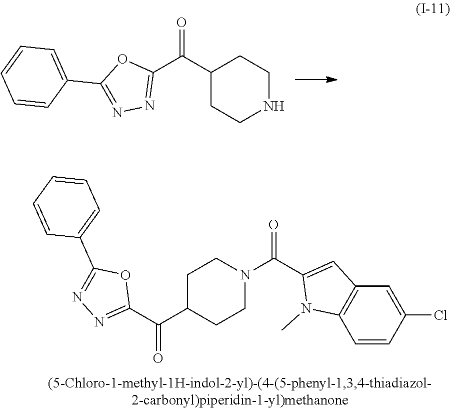 [SMILES: O=C(c1nnc(-c2ccccc2)o1)C1CCNCC1 ; Cn1c(C(=O)N2CCC(C(=O)c3nnc(-c4ccccc4)o3)CC2)cc2cc(Cl)ccc21]

Based on the synthetic method described for (5-chloro-1-methyl-1H-indol-2-yl) (4-isocyanobenzoyl)piperidin-1-yl)methanone, 16 mg of solid was obtained, with a yield of 21%. ¹H NMR (300 MHz, DMSO-d₆) δ 8.14 (dd, J=7.2, 2.3 Hz, 2H), 7.78-7.63 (m, 4H), 7.59 (dd, J=8.9, 2.4 Hz, 1H), 7.27 (dt, J=8.8, 2.5 Hz, 1H), 6.69 (s, 1H), 4.71-4.36 (m, 1H), 4.28-3.98 (m, 1H), 3.79 (d, J=2.5 Hz, 4H), 3.18-3.01 (m, 2H), 2.28-1.99 (m, 2H), 1.85-1.56 (m, 2H) ppm.

2-(Pyridin-3-yl)-1,3,4-thiadiazole

3-Pyridinecarbohydrazide (1 g, 7.3 mmol, 1 eq) was dissolved in trimethyl orthoformate, followed by the addition of p-toluenesulfonic acid (126 mg, 0.73 mmol, 0.1 eq). The mixture was refluxed at 120° C. for 1.5 hours. After completion of the reaction, it was diluted with ethyl acetate (EA), then successively washed with water and saturated brine three times each. The organic layer was dried over anhydrous sodium sulfate, and the solvent was evaporated under reduced pressure to afford a white solid weighing 915 mg, with a yield of 85%. ¹H NMR (300 MHz, DMSO-d₆) δ 9.47 (d, J=3.0 Hz, 1H), 9.22 (d, J=2.9 Hz, 1H), 8.84 (ddd, J=5.0, 3.5, 1.7 Hz, 1H), 8.43 (ddt, J=7.8, 3.8, 1.9 Hz, 1H), 7.68 (dt, J=8.0, 3.8 Hz, 1H) ppm.

4-((Hydroxy(5-(pyridin-3-yl)-1,3,4-thiadiazol-2-yl)methyl)piperidin-1-yl)methyl)butanoate

2-(Pyridin-3-yl)-1,3,4-thiadiazole (250 mg, 1.7 mmol, 1 eq) was dissolved in anhydrous THF, and then slowly added dropwise LDA (1.3 ml, 2.55 mmol, 1.5 eq) at −78° C. After stirring for 30 minutes, a solution of 1-Boc-4-piperidinemethanal (362 mg, 1.7 mmol, 1 eq) in THF was added. The reaction mixture was then warmed to room temperature and stirred for an additional 3 hours. Upon completion, the reaction mixture was quenched with saturated ammonium chloride solution, followed by extraction with EA three times. The combined organic phases were washed with saturated saline solution three times, dried over anhydrous sodium sulfate, and finally purified by column chromatography to afford a pale yellow solid (290 mg) with a yield of 47%. ¹H NMR (300 MHz, DMSO-d₆) δ 9.20 (dd, J=2.3, 0.9 Hz, 1H), 8.84 (dd, J=4.8, 1.6 Hz, 1H), 8.40 (dt, J=8.0, 2.0 Hz, 1H), 7.68 (ddd, J=7.9, 4.9, 0.9 Hz, 1H), 6.20 (d, J=5.6 Hz, 1H), 4.72 (dd, J=7.3, 5.6 Hz, 1H), 3.99 (t, J=16.7 Hz, 2H), 2.72 (s, 2H), 2.06 (ddt, J=15.3, 7.8, 3.4 Hz, 1H), 1.48 (s, 2H), 1.41 (s, 9H), 1.30-1.13 (m, 2H) ppm.

4-(5-(Pyridin-3-yl)-1,3,4-thiadiazol-2-yl)piperidine-1-carboxylate tert-butyl ester

4-(Hydroxy(5-(pyridin-3-yl)-1,3,4-thiadiazol-2-yl)methyl)piperidine-1-carboxylate tert-butyl ester (270 mg, 0.75 mmol, 1 eq) was dissolved in DCM, followed by addition of Dess-Martin periodinane (636 mg, 1.5 mmol, 2 eq). The reaction mixture was stirred at room temperature for 30 minutes. After completion of the reaction, saturated sodium bicarbonate solution was added and stirred for 5 minutes. The mixture was then extracted with EA three times, and the combined organic phases were washed with saturated sodium bicarbonate and saturated brine solution three times each. The organic layer was dried over anhydrous sodium sulfate, and the solvent was removed under reduced pressure. The resulting crude product was purified by column chromatography to afford a pale yellow solid (182 mg) with a yield of 68%. ¹H NMR (300 MHz, DMSO-d₆) δ 9.28 (dd, J=2.3, 0.9 Hz, 1H), 8.90 (dd, J=4.8, 1.7 Hz, 1H), 8.50 (dt, J=8.1, 1.9 Hz, 1H), 8.06-7.96 (m, 1H), 4.03 (d, J=13.3 Hz, 2H), 3.65 (tt, J=11.4, 3.7 Hz, 1H), 2.97 (s, 2H), 2.06-2.00 (m, 2H), 1.65-1.48 (m, 2H), 1.44 (s, 9H), 1.42-1.41 (m, 2H) ppm.

Piperidine-4-yl(5-(pyridin-3-yl)-1,3,4-thiadiazol-2-yl)methanone

182 mg of 4-(5-(Pyridin-3-yl)-1,3,4-thiadiazol-2-carbonyl)piperidine-1-carboxylate tert-butyl ester was dissolved in hydrogen chloride-diethyl ether solution. After a 30-minute reaction at room temperature, a large amount of white solid precipitated. The resulting mixture was filtered and dried, yielding 111 mg of white solid, with a yield of 84%. ¹H NMR (300 MHz, DMSO-d₆) δ 9.33 (d, J=2.3 Hz, 1H), 8.94 (dd, J=4.9, 1.6 Hz, 1H), 8.58 (dt, J=8.1, 2.0 Hz, 1H), 7.79 (dd, J=8.1, 4.9 Hz, 1H), 3.78 (tt, J=11.2, 3.7 Hz, 1H), 3.41-3.30 (m, 2H), 3.15-3.06 (m, 2H), 2.21 (dd, J=14.5, 3.6 Hz, 2H), 1.96 (qd, J=11.6, 6.0 Hz, 2H) ppm.

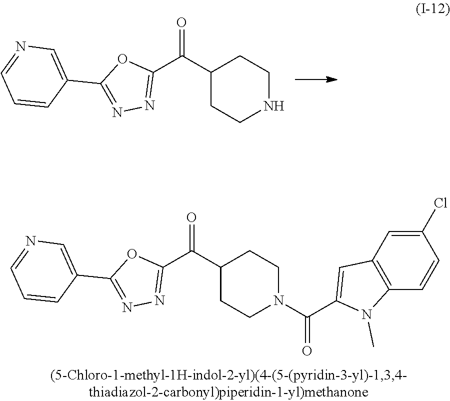 [SMILES: O=C(c1nnc(-c2cccnc2)o1)C1CCNCC1 ; Cn1c(C(=O)N2CCC(C(=O)c3nnc(-c4cccnc4)o3)CC2)cc2cc(Cl)ccc21]

Based on the synthetic method of (5-chloro-1-methyl-1H-indol-2-yl) (4-isocyanobenzyl)piperidin-1-yl)methanone, a white solid was obtained, yielding 18 mg with a yield of 11.7%. ¹H NMR (400 MHz, DMSO-d₆) δ 9.27 (dd, J=2.3, 0.9 Hz, 1H), 8.88 (dd, J=4.8, 1.7 Hz, 1H), 8.48 (dt, J=8.1, 1.9 Hz, 1H), 7.72-7.66 (m, 2H), 7.57 (d, J=8.8 Hz, 1H), 7.25 (dd, J=8.8, 2.1 Hz, 1H), 6.67 (s, 1H), 4.67-4.43 (m, 1H), 4.27-3.93 (m, 1H), 3.84-3.79 (m, 1H), 3.77 (s, 3H), 3.28-3.08 (m, 2H), 2.28-1.95 (m, 2H), 1.83-1.63 (m, 2H) ppm.

2-(3-Chlorophenyl)-1,3,4-thiadiazole

The 3-Chlorobenzoyl hydrazine (1 g, 7.3 mmol, 1 eq) was dissolved in trimethyl orthoformate, and p-toluenesulfonic acid (126 mg, 0.73 mmol, 0.1 eq) was added. The mixture was refluxed at 120° C. for 1.5 hours. After completion of the reaction, ethyl acetate (EA) was added for dilution, followed by washing with water and saturated saline solution three times each. The organic layer was dried over anhydrous sodium sulfate, and the solvent was removed under reduced pressure to afford a white solid (915 mg) with a yield of 85%. ¹H NMR (300 MHz, DMSO-d₆) δ 9.47 (d, J=3.0 Hz, 1H), 9.22 (d, J=2.9 Hz, 1H), 8.84 (ddd, J=5.0, 3.5, 1.7 Hz, 1H), 8.43 (ddt, J=7.8, 3.8, 1.9 Hz, 1H), 7.68 (dt, J=8.0, 3.8 Hz, 1H) ppm.

4-((5-(3-chlorophenyl)-1,3,4-oxadiazol-2-yl)(hydroxy)methyl)piperidine-1-carboxylic acid tert-butyl ester

2-(3-Chlorophenyl)-1,3,4-oxadiazole (250 mg, 1.7 mmol, 1 eq) was dissolved in anhydrous THF, and slowly added to LDA (1.3 ml, 2.55 mmol, 1.5 eq) at −78° C. After stirring for 30 minutes, a THF solution of 1-Boc-4-piperidinecarboxaldehyde (362 mg, 1.7 mmol, 1 eq) was added. The reaction mixture was then allowed to warm to room temperature and stirred for 3 hours. Upon completion, the reaction mixture was quenched with saturated ammonium chloride solution, followed by extraction with EA three times. The combined organic phases were washed with saturated saline solution three times, dried over anhydrous sodium sulfate, and the resulting mixture was purified by column chromatography to yield a pale yellow solid (290 mg) with a yield of 47%. ¹H NMR (300 MHz, DMSO-d₆) δ 9.20 (dd, J=2.3, 0.9 Hz, 1H), 8.84 (dd, J=4.8, 1.6 Hz, 1H), 8.40 (dt, J=8.0, 2.0 Hz, 1H), 7.68 (ddd, J=7.9, 4.9, 0.9 Hz, 1H), 6.20 (d, J=5.6 Hz, 1H), 4.72 (dd, J=7.3, 5.6 Hz, 1H), 3.99 (t, J=16.7 Hz, 2H), 2.72 (s, 2H), 2.06 (ddt, J=15.3, 7.8, 3.4 Hz, 1H), 1.48 (s, 2H), 1.41 (s, 9H), 1.30-1.13 (m, 2H) ppm.

4-(5-(3-Chlorophenyl)-1,3,4-oxadiazol-2-carbonyl)piperidine-1-carboxylic acid tert-butyl ester

The solution of 4-((5-(3-chlorophenyl)-1,3,4-oxadiazol-2-yl) (hydroxy)methyl)piperidine-1-carboxylic acid tert-butyl ester (270 mg, 0.75 mmol, 1 eq) in DCM was added with Dess-Martin periodinane (636 mg, 1.5 mmol, 2 eq) at room temperature for 30 minutes. After completion of the reaction, saturated sodium bicarbonate solution was added and stirred for 5 minutes, followed by extraction with EA three times. The combined organic phases were washed with saturated sodium bicarbonate solution and saturated saline solution three times each, dried over anhydrous sodium sulfate, and finally purified by column chromatography to afford a pale yellow solid (182 mg) with a yield of 68%. ¹H NMR (300 MHz, DMSO-d₆) δ 9.28 (dd, J=2.3, 0.9 Hz, 1H), 8.90 (dd, J=4.8, 1.7 Hz, 1H), 8.50 (dt, J=8.1, 1.9 Hz, 1H), 8.06-7.96 (m, 1H), 4.03 (d, J=13.3 Hz, 2H), 3.65 (tt, J=11.4, 3.7 Hz, 1H), 2.97 (s, 2H), 2.06-2.00 (m, 2H), 1.65-1.48 (m, 2H), 1.44 (s, 9H), 1.42-1.41 (m, 2H) ppm.

(5-(3-chlorophenyl)-1,3,4-oxadiazol-2-yl)(piperidin-4-yl)methanone

182 mg of 4-(5-(3-chlorophenyl)-1,3,4-oxadiazol-2-carbonyl)piperidine-1-carboxylic acid tert-butyl ester was dissolved in hydrogen chloride-dioxane solution, and the reaction was carried out at room temperature for 30 minutes. A large amount of white solid precipitated. After filtration and drying, a white solid weighing 111 mg was obtained, with a yield of 84%. ¹H NMR (300 MHz, DMSO-d₆) δ 9.33 (d, J=2.3 Hz, 1H), 8.94 (dd, J=4.9, 1.6 Hz, 1H), 8.58 (dt, J=8.1, 2.0 Hz, 1H), 7.79 (dd, J=8.1, 4.9 Hz, 1H), 3.78 (tt, J=11.2, 3.7 Hz, 1H), 3.41-3.30 (m, 2H), 3.15-3.06 (m, 2H), 2.21 (dd, J=14.5, 3.6 Hz, 2H), 1.96 (qd, J=11.6, 6.0 Hz, 2H) ppm.

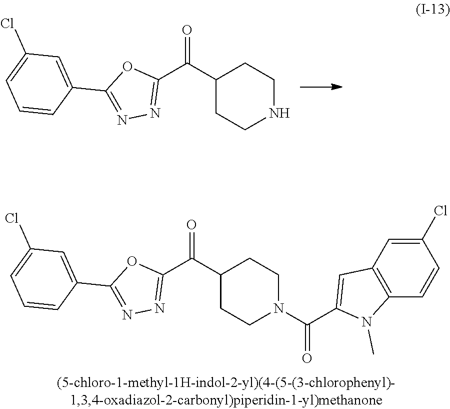 [SMILES: O=C(c1nnc(-c2cccc(Cl)c2)o1)C1CCNCC1 ; Cn1c(C(=O)N2CCC(C(=O)c3nnc(-c4cccc(Cl)c4)o3)CC2)cc2cc(Cl)ccc21]

Following the synthesis method of (5-chloro-1-methyl-1H-indol-2-yl) (4-isothiocyanatopiperidin-1-yl) methanone, a white solid weighing 18 mg was obtained with a yield of 11.7%. ¹H NMR (400 MHz, DMSO-d₆) δ 9.27 (dd, J=2.3, 0.9 Hz, 1H), 8.88 (dd, J=4.8, 1.7 Hz, 1H), 8.48 (dt, J=8.1, 1.9 Hz, 1H), 7.72-7.66 (m, 2H), 7.57 (d, J=8.8 Hz, 1H), 7.25 (dd, J=8.8, 2.1 Hz, 1H), 6.67 (s, 1H), 4.67-4.43 (m, 1H), 4.27-3.93 (m, 1H), 3.84-3.79 (m, 1H), 3.77 (s, 3H), 3.28-3.08 (m, 2H), 2.28-1.95 (m, 2H), 1.83-1.63 (m, 2H) ppm.

6-Chloropyridine-2-carbonyl hydrazide

6-Chloro-2-pyridinecarboxylic acid methyl ester (2 g, 11.66 mmol, 1 eq) was dissolved in methanol, and then hydrazine hydrate (0.7 g, 14 mmol, 1.2 eq) was added. The mixture was stirred at room temperature for 20 minutes. The solvent methanol was then removed by vacuum distillation, and MTBE was added. After stirring for 15 minutes, the mixture was filtered to obtain a white powder. The white powder was dried in a vacuum oven, yielding 1.32 g of white powder, corresponding to a yield of 66%. ¹H NMR (300 MHz, DMSO-d₆) δ 9.96 (s, 1H), 8.07 (t, J=7.7 Hz, 1H), 7.98 (dd, J=7.6, 1.1 Hz, 1H), 7.73 (dd, J=7.8, 1.1 Hz, 1H), 4.65 (s, 2H) ppm.

2-(3-Chloropyridin-2-yl)-1,3,4-oxadiazole

6-Chloropyridine-2-carbonyl hydrazide (1 g, 5.83 mmol, 1 eq) was dissolved in trimethyl orthoformate, and para-toluenesulfonic acid (100 mg, 0.58 mmol, 0.1 eq) was added. The mixture was refluxed at 120° C. for 1.5 hours. After the reaction, ethyl acetate (EA) was added for dilution, followed by washing with water and saturated saline solution three times each. The organic layer was then dried over anhydrous sodium sulfate. Finally, purification by column chromatography yielded 812 mg of white powder, with a yield of 76.73%. ¹H NMR (300 MHz, DMSO-d₆) δ 9.49 (s, 1H), 8.28-8.02 (m, 2H), 7.82 (dd, J=7.9, 1.0 Hz, 1H) ppm.

4-((Hydroxy)(5-(3-chloropyridin-2-yl)-1,3,4-oxadiazol-2-yl)methyl)piperidine-1-carboxylic acid tert-butyl ester

2-(3-Chloropyridin-2-yl)-1,3,4-oxadiazole (251 mg, 1.38 mmol, 1 eq) was dissolved in anhydrous THF, and LDA (1 ml, 2.07 mmol, 1.5 eq) was slowly added at −78° C. After stirring for 30 minutes, a THF solution of 1-Boc-4-piperidinecarboxaldehyde (295 mg, 1.38 mmol, 1 eq) was added. The reaction mixture was then allowed to warm to room temperature and stirred for 3 hours. Upon completion, the reaction mixture was quenched with saturated ammonium chloride solution, followed by extraction with EA three times. The combined organic phases were washed with saturated saline solution three times, dried over anhydrous sodium sulfate, and finally purified by column chromatography to afford a solid weighing 232.5 mg, corresponding to a yield of 42.60%. ¹H NMR (400 MHz, DMSO-d₆) δ 8.20 (dd, J=7.7, 1.0 Hz, 1H), 8.15-8.02 (m, 1H), 7.79 (dd, J=8.0, 0.9 Hz, 1H), 6.22 (d, J=5.7 Hz, 1H), 4.71 (dd, J=7.3, 5.8 Hz, 1H), 4.49-3.94 (m, 2H), 3.24 (t, J=5.8 Hz, 2H), 2.09-1.83 (m, 1H), 1.78-1.50 (m, 2H), 1.38 (s, 9H), 1.27-0.93 (m, 2H) ppm.

Piperidin-4-yl(5-(3-chloropyridin-2-yl)-1,3,4-oxadiazol-2-yl)methanone

The solution of 4-((hydroxy)(5-(3-chloropyridin-2-yl)-1,3,4-oxadiazol-2-yl)methyl) piperidine-1-carboxylic acid tert-butyl ester (220 mg, 0.56 mmol, 1 eq) in DCM was added with Dess-Martin periodinane (474 mg, 1.12 mmol, 2 eq) at room temperature for 30 minutes. After the reaction, saturated sodium bicarbonate solution was added and stirred for 5 minutes, followed by extraction with EA three times. The combined organic phases were washed with saturated sodium bicarbonate solution and saturated saline solution three times each, dried over anhydrous sodium sulfate, and finally purified by column chromatography to obtain a white powder. This solid was dissolved in hydrogen chloride-dioxane solution, and the reaction was carried out at room temperature for 30 minutes. Solid formed, was filtered, and dried to yield a pale yellow solid weighing 47.7 mg, with a yield of 26.01%. ¹H NMR (300 MHz, DMSO-d₆) δ 9.04 (s, 1H), 8.84 (s, 1H), 8.21 (d, J=7.6 Hz, 1H), 8.08 (t, J=7.8 Hz, 1H), 7.77 (d, J=8.0 Hz, 1H), 3.65 (tt, J=11.5, 3.8 Hz, 1H), 3.22 (s, 2H), 2.99 (q, J=11.7 Hz, 2H), 2.08 (d, J=13.3 Hz, 2H), 1.88-1.70 (m, 2H) ppm.

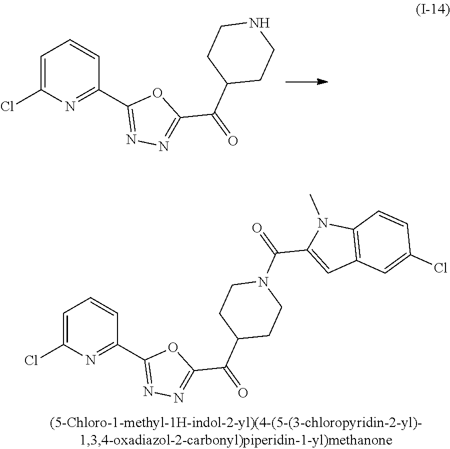 [SMILES: O=C(c1nnc(-c2cccc(Cl)n2)o1)C1CCNCC1 ; Cn1c(C(=O)N2CCC(C(=O)c3nnc(-c4cccc(Cl)n4)o3)CC2)cc2cc(Cl)ccc21]

Following the synthesis method of (5-chloro-1-methyl-1H-indol-2-yl) (4-isothiocyanatopiperidin-1-yl)methanone, a white solid weighing 43 mg was obtained with a yield of 64.95%. ¹H NMR (400 MHz, DMSO-d₆) δ 8.31 (d, J=7.7 Hz, 1H), 8.17 (t, J=7.9 Hz, 1H), 7.86 (d, J=8.0 Hz, 1H), 7.67 (d, J=2.1 Hz, 1H), 7.57 (d, J=8.8 Hz, 1H), 7.29-7.16 (m, 1H), 6.67 (s, 1H), 4.47 (d, J=14.7 Hz, 1H), 3.77 (s, 3H), 2.07 (d, J=58.7 Hz, 2H), 1.69 (qd, J=12.8, 12.0, 4.0 Hz, 2H), 1.33-1.16 (m, 4H) ppm.

2-(furan-2-yl)-1,3,4-oxadiazole

2-furancarbohydrazide (1.5 g, 11.89 mmol, 1 eq) was dissolved in trimethyl orthoformate and para-toluenesulfonic acid (200 mg, 1.2 mmol, 0.1 eq) was added. The mixture was refluxed at 120° C. for 1.5 hours. After the reaction, ethyl acetate (EA) was added for dilution, followed by washing with water and saturated saline solution three times each. The organic layer was then dried over anhydrous sodium sulfate. Finally, purification by column chromatography yielded 1.3 g of a colorless oily liquid, with a yield of 80.3%. ¹H NMR (400 MHz, DMSO-d₆) δ 9.33 (s, 1H), 8.07 (s, 1H), 7.39 (d, J=3.6 Hz, 1H), 6.81 (d, J=3.0 Hz, 1H) ppm.

4-((hydroxy)(5-(furan-2-yl)-1,3,4-oxadiazol-2-yl)methyl)piperidine-1-carboxylic acid tert-butyl ester

2-(furan-2-yl)-1,3,4-oxadiazole (1.3 g, 9.55 mmol, 1 eq) was dissolved in anhydrous THE, and LDA (7 ml, 14.33 mmol, 1.5 eq) was slowly added at −78° C. After stirring for 30 minutes, a THF solution of 1-Boc-4-piperidinecarboxaldehyde (2 g, 9.55 mmol, 1 eq) was added. The reaction mixture was then allowed to warm to room temperature and stirred for 3 hours. Upon completion, the reaction mixture was quenched with saturated ammonium chloride solution, followed by extraction with EA three times. The combined organic phases were washed with saturated saline solution three times, dried over anhydrous sodium sulfate, and finally purified by column chromatography to afford a yellow foam-like solid weighing 918.8 mg, corresponding to a yield of 27.53%. ¹H NMR (300 MHz, DMSO-d₆) δ 8.03 (d, J=1.7 Hz, 1H), 7.32 (d, J=3.5 Hz, 1H), 6.77 (dd, J=3.5, 1.8 Hz, 1H), 6.14 (d, J=5.6 Hz, 1H), 4.64 (dd, J=7.3, 5.5 Hz, 1H), 4.07-3.82 (m, 2H), 2.75-2.44 (m, 2H), 1.97 (s, 1H), 1.96-1.74 (m, 2H), 1.36 (s, 9H), 1.20-1.10 (m, 2H) ppm.

4-(5-(Furan-2-yl)-1,3,4-oxadiazol-2-carbonyl)piperidine-1-carboxylic acid tert-butyl ester

The solution of 4-((hydroxy)(5-(furan-2-yl)-1,3,4-oxadiazol-2-yl)methyl)piperidine-1-carboxylic acid tert-butyl ester (918 mg, 2.63 mmol, 1 eq) in DCM was added with Dess-Martin periodinane (2.23 g, 5.26 mmol, 2 eq) at room temperature for 30 minutes. After the reaction, saturated sodium bicarbonate solution was added and stirred for 5 minutes, followed by extraction with EA three times. The combined organic phases were washed with saturated sodium bicarbonate solution and saturated saline solution three times each, dried over anhydrous sodium sulfate, and finally purified by column chromatography to yield a white powder weighing 663 mg, with a yield of 72.62%. ¹H NMR (400 MHz, DMSO-d₆) δ 8.16 (dd, J=1.8, 0.8 Hz, 1H), 7.57 (dd, J=3.6, 0.8 Hz, 1H), 6.86 (dd, J=3.6, 1.8 Hz, 1H), 4.01 (dd, J=15.0, 9.9 Hz, 2H), 3.57 (tt, J=11.3, 3.6 Hz, 1H), 2.92 (s, 2H), 2.03-1.94 (m, 2H), 1.59-1.43 (m, 2H), 1.41 (s, 9H) ppm.

Piperidin-4-yl(5-(furan-2-yl)-1,3,4-oxadiazol-2-yl)methanone

209 mg of 4-(5-(Furan-2-yl)-1,3,4-oxadiazol-2-carbonyl)piperidine-1-carboxylic acid tert-butyl ester was dissolved in hydrogen chloride-dioxane solution and reacted at room temperature for 30 minutes. A significant amount of light yellow solid precipitated. The solid was filtered and dried to obtain 170 mg of solid, with a yield of 99.59%. ¹H NMR (400 MHz, DMSO-d₆) δ 9.07 (d, J=71.9 Hz, 2H), 8.17 (d, J=1.7 Hz, 1H), 7.59 (dd, J=3.6, 0.7 Hz, 1H), 6.87 (dd, J=3.6, 1.8 Hz, 1H), 3.71 (tt, J=11.2, 3.6 Hz, 1H), 3.33 (d, J=12.9 Hz, 2H), 3.13-3.00 (m, 2H), 2.21-2.12 (m, 2H), 1.96-1.81 (m, 2H) ppm.

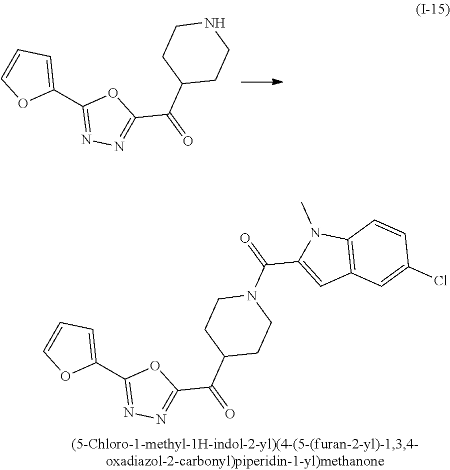 [SMILES: O=C(c1nnc(-c2ccco2)o1)C1CCNCC1 ; Cn1c(C(=O)N2CCC(C(=O)c3nnc(-c4ccco4)o3)CC2)cc2cc(Cl)ccc21]

Following the synthesis method of (5-chloro-1-methyl-1H-indol-2-yl) (4-isothiocyanatopiperidin-1-yl)methanone, a white solid weighing 70.1 mg was obtained with a yield of 75.46%. ¹H NMR (400 MHz, DMSO-d₆) δ 8.17 (d, J=1.7 Hz, 1H), 7.67 (d, J=2.1 Hz, 1H), 7.58 (s, 1H), 7.57 (d, J=13.7 Hz, 1H), 7.29-7.18 (m, 1H), 6.87 (dd, J=3.6, 1.7 Hz, 1H), 6.67 (s, 1H), 3.76 (s, 3H), 3.73 (d, J=11.9 Hz, 1H), 3.15 (s, 2H), 2.11 (s, 2H), 1.67 (qd, J=11.6, 3.9 Hz, 2H), 1.38-1.25 (m, 2H) ppm.

Following the synthesis method of (5-chloro-1-methyl-1H-indol-2-yl) (4-isothiocyanatopiperidin-1-yl)methanone, a white solid weighing 70.1 mg was obtained with a yield of 75.46%. ¹H NMR (400 MHz, DMSO-d₆) δ 8.17 (d, J=1.7 Hz, 1H), 7.67 (d, J=2.1 Hz, 1H), 7.58 (s, 1H), 7.57 (d, J=13.7 Hz, 1H), 7.29-7.18 (m, 1H), 6.87 (dd, J=3.6, 1.7 Hz, 1H), 6.67 (s, 1H), 3.76 (s, 3H), 3.73 (d, J=11.9 Hz, 1H), 3.15 (s, 2H), 2.11 (s, 2H), 1.67 (qd, J=11.6, 3.9 Hz, 2H), 1.38-1.25 (m, 2H) ppm.

2-(Thiophen-2-yl)-1,3,4-oxadiazole

2-Thiophenecarbohydrazide (1.4 g, 9.85 mmol, 1 eq) was dissolved in trimethyl orthoformate and para-toluenesulfonic acid (170 mg, 0.99 mmol, 0.1 eq) was added. The mixture was refluxed at 120° C. for 1.5 hours. After the reaction, ethyl acetate (EA) was added for dilution, followed by washing with water and saturated saline solution three times each. The organic layer was then dried over anhydrous sodium sulfate. Finally, purification by column chromatography yielded 1.2 g of a colorless oily liquid, with a yield of 80.08%. ¹H NMR (400 MHz, DMSO-d₆) δ 9.30 (s, 1H), 7.96 (dd, J=5.0, 1.2 Hz, 1H), 7.91-7.82 (m, 1H), 7.30 (dd, J=5.0, 3.7 Hz, 1H) ppm.

4-((Hydroxy)(5-(thiophen-2-yl)-1,3,4-oxadiazol-2-yl)methyl)piperidine-1-carboxylic acid tert-butyl ester

2-(Thiophen-2-yl)-1,3,4-oxadiazole (1.2 g, 7.89 mmol, 1 eq) was dissolved in anhydrous THF, and LDA (6 ml, 11.84 mmol, 1.5 eq) was slowly added at −78° C. After stirring for 30 minutes, a THF solution of 1-Boc-4-piperidinecarboxaldehyde (1.7 g, 7.89 mmol, 1 eq) was added. The reaction mixture was then allowed to warm to room temperature and stirred for 3 hours. Upon completion, the reaction mixture was quenched with saturated ammonium chloride solution, followed by extraction with EA three times. The combined organic phases were washed with saturated saline solution three times, dried over anhydrous sodium sulfate, and finally purified by column chromatography to afford a yellow foam-like solid weighing 552.5 mg, corresponding to a yield of 19.17%. ¹H NMR (400 MHz, DMSO-d₆) δ 7.95 (dd, J=5.0, 1.2 Hz, 1H), 7.82 (dd, J=3.7, 1.2 Hz, 1H), 7.29 (dd, J=5.0, 3.7 Hz, 1H), 6.16 (d, J=5.6 Hz, 1H), 4.65 (dd, J=7.4, 5.6 Hz, 1H), 4.06-3.90 (m, 2H), 2.69 (s, 2H), 2.01 (ddd, J=11.8, 5.6, 2.3 Hz, 1H), 1.93-1.41 (m, 1H), 1.38 (s, 9H), 1.18 (dtt, J=20.5, 12.6, 6.2 Hz, 2H) ppm.

4-(5-(Thiophen-2-yl)-1,3,4-oxadiazol-2-carbonyl)piperidine-1-carboxylic acid tert-butyl ester

A solution of 4-((hydroxy)(5-(thiophen-2-yl)-1,3,4-oxadiazol-2-yl)methyl)piperidine-1-carboxylic acid tert-butyl ester (552 mg, 1.51 mmol, 1 eq) in DCM was added with Dess-Martin periodinane (1.3 g, 3.02 mmol, 2 eq) at room temperature for 30 minutes. After the reaction, saturated sodium bicarbonate solution was added and stirred for 5 minutes, followed by extraction with EA three times. The combined organic phases were washed with saturated sodium bicarbonate solution and saturated saline solution three times each, dried over anhydrous sodium sulfate, and finally purified by column chromatography to yield a yellow solid weighing 496.6 mg, with a yield of 90.46%. ¹H NMR (400 MHz, DMSO-d₆) δ 8.07 (dd, J=5.0, 1.2 Hz, 1H), 7.99 (dd, J=3.8, 1.2 Hz, 1H), 7.35 (dd, J=5.0, 3.8 Hz, 1H), 3.99 (d, J=13.2 Hz, 2H), 3.57 (dq, J=11.3, 3.9 Hz, 2H), 3.17 (s, 1H), 2.03-1.94 (m, 2H), 1.50 (td, J=12.4, 4.2 Hz, 2H), 1.41 (s, 9H) ppm.

Piperidin-4-yl(5-(thiophen-2-yl)-1,3,4-oxadiazol-2-yl)methanone

277 mg of 4-(5-(thiophen-2-yl)-1,3,4-oxadiazol-2-carbonyl)piperidine-1-carboxylic acid tert-butyl ester was dissolved in hydrogen chloride-dioxane solution and reacted at room temperature for 30 minutes, yielding a significant amount of light yellow solid precipitate. After filtration and drying, a light yellow solid weighing 195 mg was obtained, with a yield of 85.35%. ¹H NMR (400 MHz, DMSO-d₆) δ 9.13 (s, 1H), 8.94 (s, 1H), 8.09 (dd, J=5.0, 1.2 Hz, 1H), 8.00 (dd, J=3.8, 1.2 Hz, 1H), 7.36 (dd, J=5.0, 3.8 Hz, 1H), 3.71 (tt, J=11.2, 3.6 Hz, 1H), 3.31 (s, 2H), 3.18-2.97 (m, 2H), 2.16 (dd, J=14.2, 3.6 Hz, 2H), 2.02-1.81 (m, 2H) ppm.

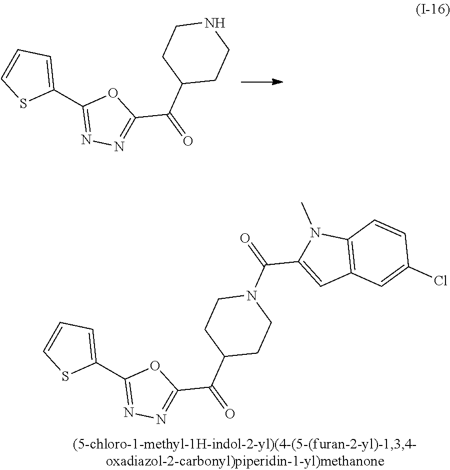 [SMILES: O=C(c1nnc(-c2cccs2)o1)C1CCNCC1 ; Cn1c(C(=O)N2CCC(C(=O)c3nnc(-c4cccs4)o3)CC2)cc2cc(Cl)ccc21]

Following the synthesis method of (5-chloro-1-methyl-1H-indol-2-yl) (4-isothiocyanatopiperidin-1-yl)methanone, a pale yellow solid weighing 90 mg was obtained with a yield of 58.69%. ¹H NMR (400 MHz, DMSO-d₆) δ 8.08 (dd, J=5.0, 1.2 Hz, 1H), 8.00 (dd, J=3.8, 1.2 Hz, 1H), 7.67 (d, J=2.0 Hz, 1H), 7.57 (d, J=8.8 Hz, 1H), 7.35 (dd, J=5.0, 3.7 Hz, 1H), 7.29-7.16 (m, 1H), 6.67 (d, J=0.9 Hz, 1H), 4.51-4.03 (m, 2H), 3.76 (s, 3H), 3.75-3.65 (m, 1H), 3.14 (s, 1H), 2.11 (s, 2H), 1.74-1.59 (m, 2H) ppm.

6-Methoxypyridine-2-carbohydrazide

6-Methoxy-2-pyridinecarboxylic acid methyl ester (1.04 g, 6.22 mmol, 1 eq) was dissolved in methanol, then hydrated hydrazine (374 mg, 7.46 mmol, 1.2 eq) was added. The mixture was stirred at room temperature for 20 minutes. After that, the solvent methanol was removed by vacuum distillation. Then, MTBE was added and stirred for 15 minutes. The mixture was filtered to obtain a white powder-like solid, which was further dried in a vacuum oven. The yield of the white powder was 914 mg, corresponding to 87.88%. ¹H NMR (400 MHz, DMSO-d₆) δ 9.75 (d, J=16.0 Hz, 1H), 7.90-7.77 (m, 1H), 7.55 (dt, J=17.7, 6.8 Hz, 1H), 6.97 (q, J=9.1, 8.5 Hz, 1H), 4.57 (t, J=10.4 Hz, 2H), 4.04-3.84 (m, 3H) ppm.

2-(3-Methoxypyridin-2-yl)-1,3,4-oxadiazole

6-Methoxypyridine-2-carbohydrazide (893 mg, 5.34 mmol, 1 eq) was dissolved in trimethyl orthoformate. To this solution, 92 mg of p-toluenesulfonic acid (0.53 mmol, 0.1 eq) was added. The mixture was refluxed at 120° C. for 1.5 hours. After completion of the reaction, it was diluted with ethyl acetate (EA), followed by washing with water and saturated saline solution three times each. The organic layer was dried over anhydrous sodium sulfate. Finally, the crude product was purified by column chromatography to obtain a white powder solid weighing 796.7 mg with a yield of 84.18%. ¹H NMR (400 MHz, DMSO-d₆) δ 9.40 (s, 1H), 7.95 (dd, J=8.3, 7.3 Hz, 1H), 7.81 (dd, J=7.3, 0.8 Hz, 1H), 7.09 (dd, J=8.4, 0.8 Hz, 1H), 3.95 (s, 3H) ppm.

4-(Hydroxy(5-(3-methoxypyridin-2-yl)-1,3,4-oxadiazol-2-yl)methyl)piperidine-1-carboxylate

2-(3-Methoxypyridin-2-yl)-1,3,4-oxadiazole (251 mg, 1.42 mmol, 1 eq) was dissolved in anhydrous THF, followed by slow addition of LDA (1.4 mL, 2.84 mmol, 2 eq) at −78° C., and the mixture was stirred for 30 minutes. Then, a solution of 1-Boc-4-piperidone (302 mg, 1.42 mmol, 1 eq) in THF was added. The reaction mixture was allowed to warm to room temperature and stirred for 3 hours. After completion of the reaction, it was quenched with saturated ammonium chloride solution, and then extracted with EA three times. The organic layers were combined, washed with saturated saline solution three times, dried over anhydrous sodium sulfate, and finally purified by column chromatography to yield a yellow oily liquid of 248.5 mg, with a yield of 44.92%. ¹H NMR (400 MHz, DMSO-d₆) δ 7.94 (dt, J=8.6, 7.0 Hz, 1H), 7.77 (d, J=7.3 Hz, 1H), 7.09 (d, J=8.3 Hz, 1H), 6.18 (d, J=5.6 Hz, 1H), 4.74-4.62 (m, 1H), 4.47 (t, J=5.3 Hz, 2H), 3.92 (s, 3H), 3.24 (t, J=5.7 Hz, 2H), 1.87 (d, J=13.3 Hz, 1H), 1.66-1.57 (m, 2H), 1.39 (d, J=1.9 Hz, 9H), 1.27-1.20 (m, 2H) ppm.

4-(5-(3-Methoxypyridin-2-yl)-1,3,4-oxadiazol-2-yl)piperidine-1-carboxylate

The solution of 4-(hydroxy(5-(3-methoxypyridin-2-yl)-1,3,4-oxadiazol-2-yl)methyl) piperidine-1-carboxylate tert-butyl ester (220 mg, 0.62 mmol, 1 eq) in DCM was treated with Dess-Martin periodinane (522 mg, 1.24 mmol, 2 eq) at room temperature for 30 minutes. After completion of the reaction, the mixture was diluted with saturated sodium bicarbonate solution and stirred for 5 minutes. The combined organic phases were extracted with EA three times, followed by washing with saturated sodium bicarbonate solution and saturated saline water three times each. The organic layer was dried over anhydrous sodium sulfate, and the resulting mixture was subjected to column chromatography to afford a yellow oily droplet of 149.1 mg, with a yield of 62.45%. ¹H NMR (400 MHz, DMSO-d₆) δ 7.99 (dd, J=8.3, 7.3 Hz, 1H), 7.91 (dd, J=7.4, 0.9 Hz, 1H), 7.15 (dd, J=8.3, 0.9 Hz, 1H), 3.97 (s, 3H), 3.61 (tt, J=11.4, 3.6 Hz, 1H), 2.07-1.96 (m, 2H), 1.83-1.78 (m, 2H), 1.51 (qd, J=12.1, 4.5 Hz, 2H), 1.40 (d, J=9.5 Hz, 9H), 1.29-1.23 (m, 2H) ppm.

N-((5-(3-Methoxypyridin-2-yl)-1,3,4-oxadiazol-2-yl)methyl)piperidine-4-carboxamide

140 mg of 4-((5-(3-Methoxypyridin-2-yl)-1,3,4-oxadiazol-2-yl)carbonyl)piperidine-1-carboxylic acid tert-butyl ester was dissolved in hydrogen chloride-dioxane solution, and the reaction proceeded at room temperature for 30 minutes. A large amount of pale yellow solid precipitated out, which was then filtered and dried to yield 112 mg of yellow solid, with a yield of 95.68%. ¹H NMR (400 MHz, DMSO-d₆) δ 9.59 (s, 1H), 9.12 (s, 1H), 8.05-7.96 (m, 1H), 7.92 (d, J=7.2 Hz, 1H), 7.16 (d, J=8.3 Hz, 1H), 3.97 (s, 3H), 3.38-3.20 (m, 2H), 3.15-3.02 (m, 2H), 2.24-2.13 (m, 2H), 2.04-1.97 (m, 1H), 1.91 (dtd, J=15.4, 11.9, 4.1 Hz, 2H) ppm.

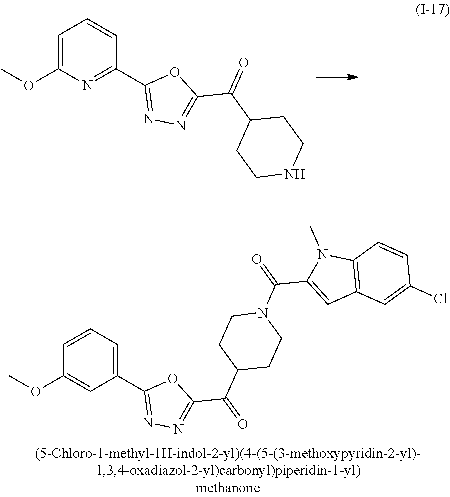 [SMILES: COc1cccc(-c2nnc(C(=O)C3CCNCC3)o2)n1 ; COc1cccc(-c2nnc(C(=O)C3CCN(C(=O)c4cc5cc(Cl)ccc5n4C)CC3)o2)c1]

Following the synthesis method of (5-chloro-1-methyl-1H-indol-2-yl) (4-isobutanoylpiperidin-1-yl)methanone, a pale yellow solid was obtained, yielding 111.8 mg, with a yield of 72.75%. ¹H NMR (400 MHz, DMSO-d₆) δ 7.98 (d, J=8.0 Hz, 1H), 7.92 (d, J=7.2 Hz, 1H), 7.67 (d, J=2.0 Hz, 1H), 7.56 (s, 1H), 7.49-7.29 (m, 1H), 7.29-7.24 (m, 1H), 6.65 (d, J=18.0 Hz, 2H), 3.97 (s, 3H), 3.77 (s, 3H), 3.28 (s, 2H), 2.90 (s, 2H), 2.69 (s, 1H), 1.69 (qd, J=12.0, 4.2 Hz, 2H), 1.60-1.44 (m, 2H) ppm.

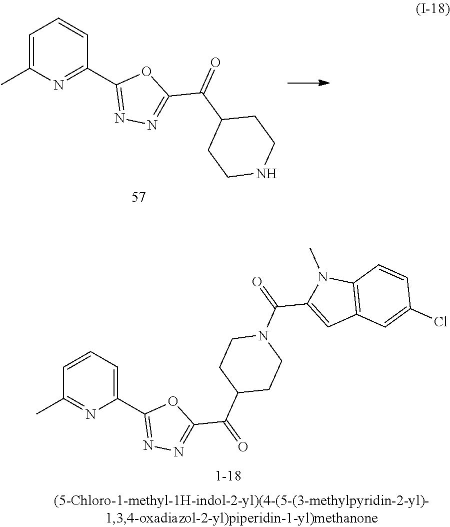 [SMILES: Cc1cccc(-c2nnc(C(=O)C3CCNCC3)o2)n1 ; Cc1cccc(-c2nnc(C(=O)C3CCN(C(=O)c4cc5cc(Cl)ccc5n4C)CC3)o2)n1]

Based on the synthesis method of I-1, a white powder solid was obtained, yielding 47 mg, with a yield of 36.37%. ¹H NMR (400 MHz, DMSO-d₆) δ 7.75 (t, J=2.9 Hz, 1H), 7.63 (d, J=5.4 Hz, 1H), 7.59-7.52 (m, 1H), 7.33 (dd, J=9.3, 4.0 Hz, 1H), 7.24 (d, J=9.0 Hz, 1H), 7.19 (d, J=3.7 Hz, 1H), 6.69-6.58 (m, 1H), 4.02 (s, 3H), 3.76 (dd, J=19.6, 3.8 Hz, 4H), 2.58 (d, J=0.7 Hz, 3H), 2.36 (p, J=6.4 Hz, 1H), 1.85-1.48 (m, 4H) ppm.

### Example 2: In Vitro FAAH Enzyme Inhibition Activity Assay

FAAH hydrolyzes AEA to form ethanolamine within cells. Different enzyme activities catalyze the formation of varying amounts of products from a fixed amount of substrate. The enzyme activity level can be assessed by measuring the amount of products generated. Based on this principle, experiments were designed in this invention. According to the FAAH assay kit instructions, a certain concentration of FAAH was diluted and added to a buffer solution along with a fluorescently labeled substrate. Compounds of various concentrations were then added, along with blank control groups and JZL-195 positive control groups. After the reaction, fluorescence analysis was performed using a plate reader, and the inhibition rate was calculated.

The specific results are shown in the table:

| Compound name | IC₅₀ | |
|---|---|---|
| Control: JZL-195 | 2.810 | nm |
| I-1 | 27.92 | μm |
| I-2 | 14.24 | μm |
| I-3 | 13.48 | μm |
| I-4 | 10.06 | μm |
| I-5 | 125.0 | μm |
| I-6 | 131.9 | μm |
| I-7 | 77.90 | nm |
| I-8 | 1.029 | μm |
| I-9 | 279.1 | nm |
| I-10 | 112.0 | nm |
| I-11 | 16.81 | nm |
| I-12 | 28.55 | nm |
| 1-13 | 89.67 | nm |
| I-14 | 6.21 | nm |
| I-15 | 3.11 | nm |
| I-16 | 12.94 | nm |
| I-17 | 38.12 | nm |
| I-18 | 49.86 | nm |

As shown in the table above, the compounds of the present invention exhibit excellent FAAH inhibitory activity.

### Example 3

### Anti-Neuroinflammatory Activity Testing of Compound I-11

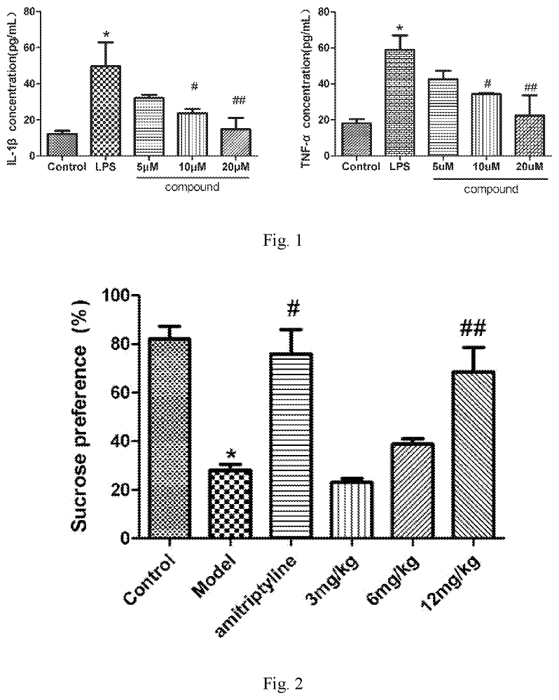

The neuroinflammatory model was established using BV2 cells treated with LPS, and the inhibitory effect of compound I-11 on neuroinflammation was investigated by measuring the release levels of inflammatory cytokines (TNF-α, IL-1β). As shown in FIG. 1 and Table 1, the expression levels of inflammatory cytokines (TNF-α, IL-1β) were extremely low in the Control group cells. After LPS treatment, the expression levels of TNF-α and IL-1β significantly increased (p<0.01).

However, pretreatment with compound I-11 resulted in a dose-dependent reduction in the expression of inflammatory cytokines TNF-α and IL-1β in the cells. At high concentrations (20 μM), the compound was able to restore the expression of inflammatory cytokines to near-normal levels. These experimental results indicate that compound 1 effectively inhibits the LPS-induced inflammatory response in BV2 cells, demonstrating promising potential for further development as an anti-neuroinflammatory agent.

TABLE 1
Anti-inflammatory activity assay of compound I-11

| Group | IL-1β(pg/mL) | TNF-α(pg/mL) |
|---|---|---|
| Control | 15.3 ± 2.2 | 19.7 ± 4.4 |
| Model | 49.4 ± 7.3* | 59.7 ± 1.7* |
| 1(5 μM) | 33.7 ± 4.8^{##} | 42.4 ± 3.1^{##} |
| 1 (10 μM) | 23.6 ± 3.6 | 36.2 ± 5.5 |
| 1 (20 μM) | 18.7 ± 2.2^{#} | 22.4 ± 6.2^{#} |

*p < 0.01 vs control group,
^{#}p < 0.05 vs LPS group,
^{##}p < 0.01 vs LPS group.

Using the acetic acid writhing test in mice as a pain model to evaluate the analgesic activity of compound I-11. The experimental animal information and procedures are outlined below.

### a) Experimental Animals

Healthy ICR mice, of clean grade, weighing 20-25 g. The animal facility complies with the requirements of a clean laboratory, with a temperature of 20-22° C., humidity of 40%˜70%, and controlled lighting on a 12-hour light/12-hour dark cycle (lighting from 8:00 to 20:00). Mice are housed in plastic cages (280×150×180 mm) with 10 mice per cage, separated by gender, and provided ad libitum access to food and water. The diet consists of experimental rodent pellets, and the drinking water is city tap water sterilized by high-temperature autoclaving. Mice are allowed a 7-day acclimatization period. All procedures involving animals are conducted following the guidelines of the International Association for the Study of Pain Ethics Committee. Mice are housed in the animal facility upon purchase.

### b) Experimental Drugs and Reagents

Glacial acetic acid and physiological saline were obtained from the China Pharmaceutical University reagent repository. Compound I-11 was a laboratory-synthesized compound (purity>98%).

### c) Experimental Procedure

Mice were marked with saturated bitter solution, with the left front limb marked as number 1, left hind limb as number 2, right front limb as number 3, right hind limb as number 4, and head as number 5. Combinations such as head with left front limb as number 6, head with left hind limb as number 7, head with right front limb as number 8, head with right hind limb as number 9, and tail as number 10 were used for identification. Animals were divided into groups, with n=10 in each group, evenly distributed between males and females.

The experiment employed the acetic acid-induced writhing test in mice to investigate the analgesic activity of the samples. Kunming mice weighing 20±2 g, with an equal number of males and females, were randomly divided into four groups, each consisting of 10 mice. These groups included high, medium, and low dose groups of the sample, as well as a control group. The sample was subcutaneously administered to mice at high (5 mg·kg⁻¹), medium (2.5 mg·kg⁻¹), and low (1.25 mg kg⁻¹) doses, while the control group received an equivalent volume of physiological saline (0.9% NaCl). After 0.5 hours of administration, each mouse was intraperitoneally injected with 10 mL/kg of 0.7% glacial acetic acid. The number of writhing movements and the latency period of mice were observed and recorded 15 minutes after acetic acid injection, and the analgesic percentage was calculated. Statistical analysis was performed using the t-test to determine significance.

Analgesic percentage (%)=(Mean writhing count of control group-Mean writhing count of treated group)/Mean writhing count of control group

### Experimental Results Description:

Based on the acetic acid-induced writhing test in mice, the writhing counts of mice in the blank control group, morphine group, and I-11 group were compared within 15 minutes. At a dose of 5 mg·kg⁻¹, I-11 exhibited a writhing inhibition rate of 87.1% in mice, indicating that compound I-11 possesses significant analgesic activity.

The inhibitory effect of compound I-11 at different concentrations on the writhing count in mice.

| | Does mg/kg | | latency | |
|---|---|---|---|---|
| Compound | (s.c) | writhing count | period (s) | Inhibition |
| Control | / | 43.4 ± 1.65 ** | 3.9 ± 0.57 ** | — |
| Morphine | 5 | 0 | 0 | 100.0% |
| Low dose | 1.25 | 25.9 ± 1.37 ** | 5.4 ± 0.7 ** | 40.3% |
| Medium | 2.5 | 16.0 ± 1.7 ** | 5.7 ± 0.82 ** | 63.1% |
| dose | | | | |
| High dose | 5 | 5.6 ± 1.26 ** | 5.8 ± 0.92 ** | 87.1% |

### Compound I-11 Anti-depression Activity Study

Male SD rats weighing between 180 g to 200 g were randomly divided into 6 groups, with 8 rats in each group, as follows:

Normal group; Chlorpromazine group; Positive control group (Amitriptyline, 6 mg/kg); I-11 low dose group (3 mg/kg); I-11 medium dose group (6 mg/kg); I-11 high dose group (12 mg/kg);

In the normal group, rats were maintained under normal conditions. In other groups, rats were intraperitoneally injected with chlorpromazine (0.2 mg/kg) once a day for three days to induce the model. After successful modeling, the respective drugs were administered according to the group. After two weeks of continuous administration, sucrose consumption test, open field test, and forced swimming test were conducted sequentially.

### 1.1 Sucrose Preference Test

The American Psychiatric Association considers a significant feature of depression to be the decrease in reward responsiveness, indicating a loss of pleasure. The sucrose preference test is a well-established measure for assessing reward responsiveness in experimental animals.

As shown in FIG. 2 and Table 2, compared to the control group, the sucrose intake of the model group significantly decreased (p<0.01), indicating a pronounced loss of pleasure in the model rats and confirming the successful modeling of depression. In comparison to the model group, the positive control group (treated with amitriptyline) showed a significant increase in sucrose intake, indicating a substantial alleviation of depressive symptoms. Additionally, compared to the model group, the high-dose I-11 group exhibited a significant difference (p<0.01), albeit slightly less effective than the positive control drug amitriptyline, suggesting that compound I-11 possesses certain antidepressant effects.

TABLE 2
Sucrose Preference Test Study

| Group | Sucrose intake ratio (%) |
|---|---|
| Control | 83.7 ± 6.6 |
| Model | 25.4 ± 1.7* |
| Amitriptyline | 78.6 ± 4.6^{##} |
| 1 (3 mg/kg) | 20.8 ± 20.1 |
| 1 (6 mg/kg) | 38.3 ± 6.7^{#} |
| 1 (12 mg/kg) | 70.7 ± 9.6^{##} |

*p < 0.01 vs control group,
^{#}p < 0.01 vs Model group.

### 1.2 Open Field Test

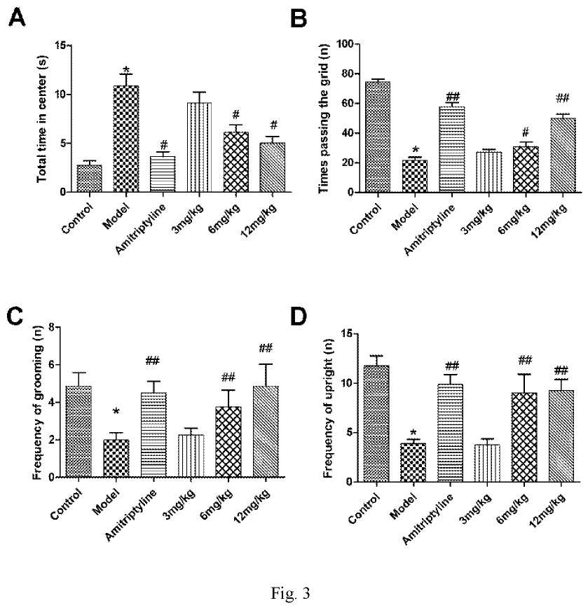

After the sucrose preference test, we conducted an open field test on the experimental animals. The results, as shown in FIG. 3 and Table 3, revealed that compared to the normal group, the central time spent by rats in the open field test significantly increased in the model group (p<0.01). Additionally, the number of grid crossings, rearing frequency, and grooming frequency all significantly decreased (p<0.01). Notably, in terms of grid crossings, rearing frequency, and grooming frequency, the high-dose I-11 group showed efficacy comparable to the positive control amitriptyline group, indicating a clear antidepressant effect of I-11.

TABLE 3
Open Field Test Study

| | Total time | Times passing | Frequency | Frequency |
|---|---|---|---|---|
| Group | in center (s) | the grid (n) | of grooming (n) | of upright (n) |
| Control | 3.8 ± 0.6 | 79.1 ± 2.6 | 4.7 ± 0.1 | 13.1 ± 6.6 |
| Model | 12.4 ± 1.7* | 22.3 ± 3.7* | 2.0 ± 0.7* | 4.4 ± 0.1* |
| Amitriptyline | 4.6 ± 0.6^{##} | 58.9 ± 2.2^{##} | 4.3 ± 0.3^{##} | 10.6 ± 2.2^{##} |
| 1 (3 mg/kg) | 8.8 ± 0.1 | 23.8 ± 2.1 | 2.2 ± 0.6 | 5.5 ± 0.7 |
| 1 (6 mg/kg) | 6.3 ± 0.7^{#} | 28.3 ± 3.3^{#} | 3.3 ± 0.7^{#} | 8.3 ± 2.3^{#} |
| 1 (12 mg/kg) | 5.7 ± 1.2^{##} | 50.8 ± 3.6^{##} | 4.4 ± 0.3^{##} | 8.7 ± 1.6^{##} |

*P value compared to control group (*P < 0.01);
^{#}P value compared to model group (^{#}P < 0.05, ^{##}P < 0.01).

### 1.3 Forced Swimming Test

The results of the Forced Swimming Test showed that compared to the control group, the immobility time of rats in the model group was significantly prolonged (p<0.01). Following drug intervention, both the Amitriptyline group and the I-11 medium and high dose groups were able to reduce the immobility time of the rats in the LPS model (p<0.05). Among them, the effect of the I-11 high dose (12 mg/kg) group was slightly lower than that of the Amitriptyline group (6 mg/kg), indicating that Compound 1 has a certain potential for antidepressant effects. The results are shown in Table 4 below.

TABLE 4
Results of Forced Swimming Test

| Group | Immobility Time (s) |
|---|---|
| Control | 75.67 ± 7.30 |
| Model | 125.64 ± 10.17* |
| Amitriptyline | 88.47 ± 4.64^{##} |
| 1 (3 mg/kg) | 110.66 ± 10.31 |
| 1 (6 mg/kg) | 100.73 ± 6.69^{#} |
| 1 (12 mg/kg) | 95.44 ± 6.97^{##} |

*P value compared to control group (*P < 0.01);
^{#}P value compared to model group (^{#}P < 0.05, ^{##}P < 0.01).

The above-described embodiments are merely preferred implementations of the present invention. It should be noted that for those skilled in the art, various modifications and refinements can be made without departing from the principles of the present invention. These modifications and refinements should also be considered within the scope of the present invention's protection.

1. A compound as shown in formula I or a pharmaceutically acceptable salt thereof: 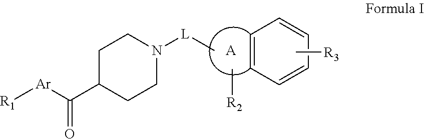
wherein,
Ar is an unsubstituted, monosubstituted, or polysubstituted heteroaromatic ring; wherein the heteroaromatic ring is selected from: furan, thiophene, pyrrole, oxazole, isoxazole, imidazole, pyrazole, thiazole, isothiazole, triazole, 1,3,4-oxadiazole, 1,3,4-thiadiazole, 1,2,4-oxadiazole, 1,2,4-thiadiazole, pyridine, pyrimidine, pyridazine, benzoxazole, benzothiazole, purine, quinoline, isoquinoline, indazole, oxazolo[4,5-b]pyridine, and oxazolo[5,4-b]pyridine;
R₁ represents H or aryl or heteroaryl;
L represents a linker, selected from —O—, —S—, —NR₄—, 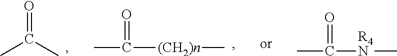
n=0, 1, 2, 3;
R₄ is selected from H, C₁-C₈ straight or branched halogenated alkyl, halogenated cycloalkyl, C₁-C₈ straight or branched alkyl, cycloalkyl; 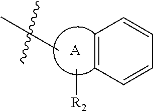
is selected from 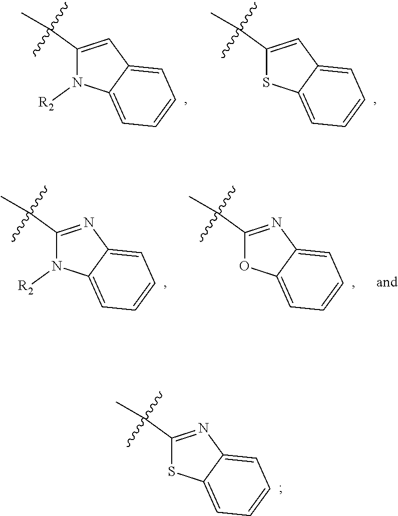
R₂ is selected from H, methyl, ethyl, acetyl, phenyl, pyridyl, methoxycarbonyl, propionyl, methoxyacetyl, and ethoxyacetyl;
R₃ is selected fromH, F, Cl, Br, I, CF₃, CN, hydroxyl, methoxy, methyl, nitro, thiol, carboxyl, vinyl, bromomethyl, cyanomethyl, aryl, heterocyclyl, C₁-C₁₀ straight or branched alkyl, cycloalkyl, C₁-C₁₀ straight or branched alkyl substituted thiol group, C₁-C₁₀ straight or branched alkyl ester group.

2. (canceled)

3. The compound according to claim 1, wherein, the heteroaromatic ring is selected from the following groups:

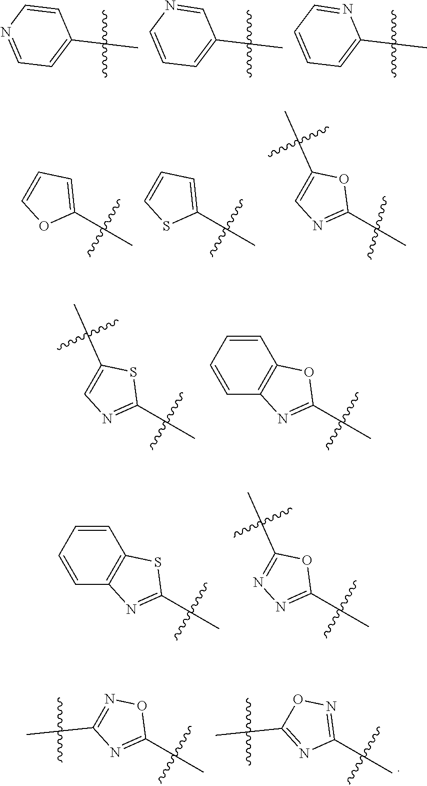

4. The compound according to claim 1, wherein, R₁ represents H, aryl or heteroaryl; wherein the aryl or heteroaryl is unsubstituted, monosubstituted, or polysubstituted, the aryl or heteroaryl is selected from phenyl, furanyl, thiophenyl, pyrrolyl, oxazolyl, isooxazolyl, imidazolyl, pyrazolyl, thiazolyl, isothiazolyl, triazolyl, 1,3,4-oxadiazolyl, 1,3,4-thiadiazolyl, 1,2,4-oxadiazolyl, 1,2,4-thiadiazolyl, pyridinyl, pyrimidinyl, pyridazinyl, benzofuranyl, indolyl, quinolinyl, isoquinolinyl, indazolyl, benzoxazolyl, benzothiazolyl, purinyl, oxazolopyridinyl; the substituent on the aryl or the heteroaryl is selected from H, F, Cl, Br, I, CF₃, CN, hydroxyl, methoxy, methyl, nitro, thiol, carboxyl, vinyl, bromomethyl, cyanomethyl, C₁-C₁₀ straight or branched alkoxy, C₁-C₁₀ straight or branched halogenated alkyl, halogenated cycloalkyl, C₁-C₁₀ straight or branched alkyl, cycloalkyl, C₁-C₁₀ straight or branched alkyl substituted thiol group, C₁-C₁₀ straight or branched alkyl amide group, C₁-C₁₀ straight or branched alkyl ester group.

5. The compound according to claim 4, wherein,
R₁ is selected from 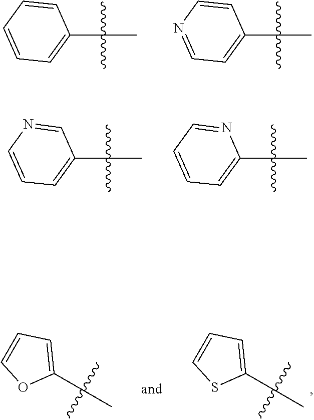 [SMILES: *c1ccccc1 ; *c1ccncc1 ; *c1cccnc1 ; *c1ccccn1 ; *c1ccco1 ; *c1cccs1]
the substituent on R₁ is selected from H, F, Cl, Br, I, methoxy, and methyl.

6. The compound according to claim 1, wherein,
L is —CO—;
and/or, R₂ is selected from H, and methyl;
and/or, R₃ is selected from H, F, Cl, Br, and I.

7. The compound according to claim 1, wherein, 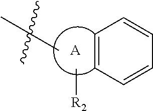
is selected from

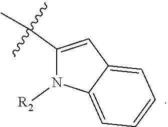

8. A compound or a pharmaceutically acceptable salt thereof, selected from:

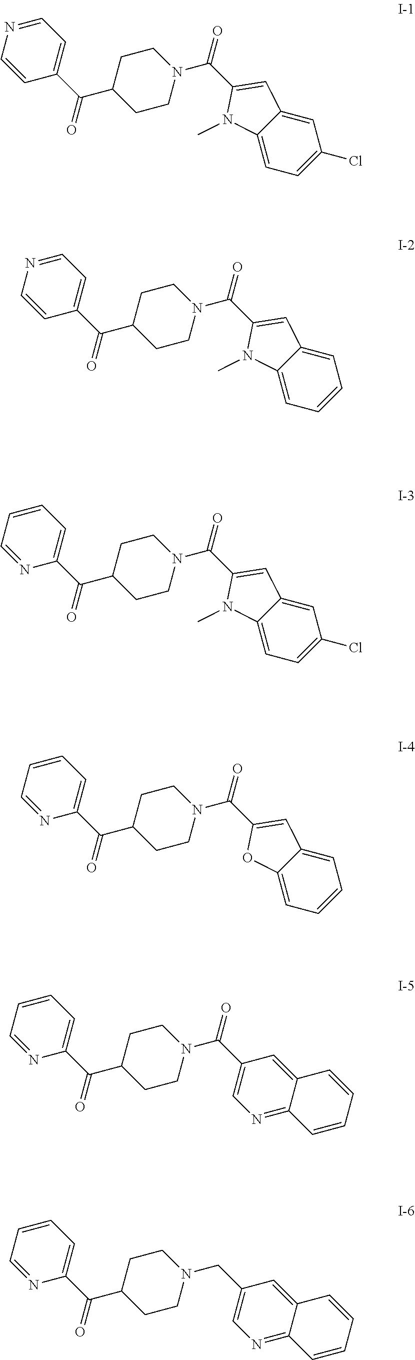 [SMILES: Cn1c(C(=O)N2CCC(C(=O)c3ccncc3)CC2)cc2cc(Cl)ccc21 ; Cn1c(C(=O)N2CCC(C(=O)c3ccncc3)CC2)cc2ccccc21 ; Cn1c(C(=O)N2CCC(C(=O)c3ccccn3)CC2)cc2cc(Cl)ccc21 ; O=C(c1ccccn1)C1CCN(C(=O)c2cc3ccccc3o2)CC1 ; O=C(c1ccccn1)C1CCN(C(=O)c2cnc3ccccc3c2)CC1 ; O=C(c1ccccn1)C1CCN(Cc2cnc3ccccc3c2)CC1]

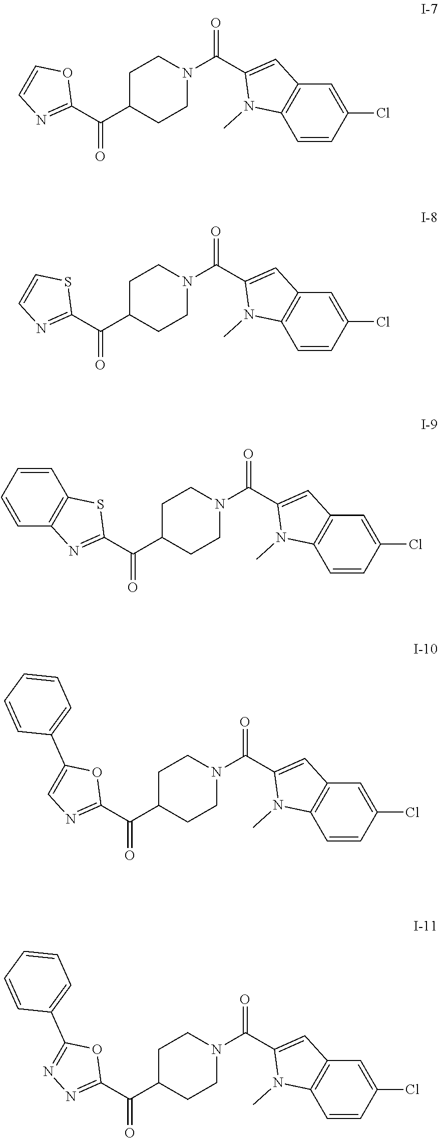 [SMILES: Cn1c(C(=O)N2CCC(C(=O)c3ncco3)CC2)cc2cc(Cl)ccc21 ; Cn1c(C(=O)N2CCC(C(=O)c3nccs3)CC2)cc2cc(Cl)ccc21 ; Cn1c(C(=O)N2CCC(C(=O)c3nc4ccccc4s3)CC2)cc2cc(Cl)ccc21 ; Cn1c(C(=O)N2CCC(C(=O)c3ncc(-c4ccccc4)o3)CC2)cc2cc(Cl)ccc21 ; Cn1c(C(=O)N2CCC(C(=O)c3nnc(-c4ccccc4)o3)CC2)cc2cc(Cl)ccc21]

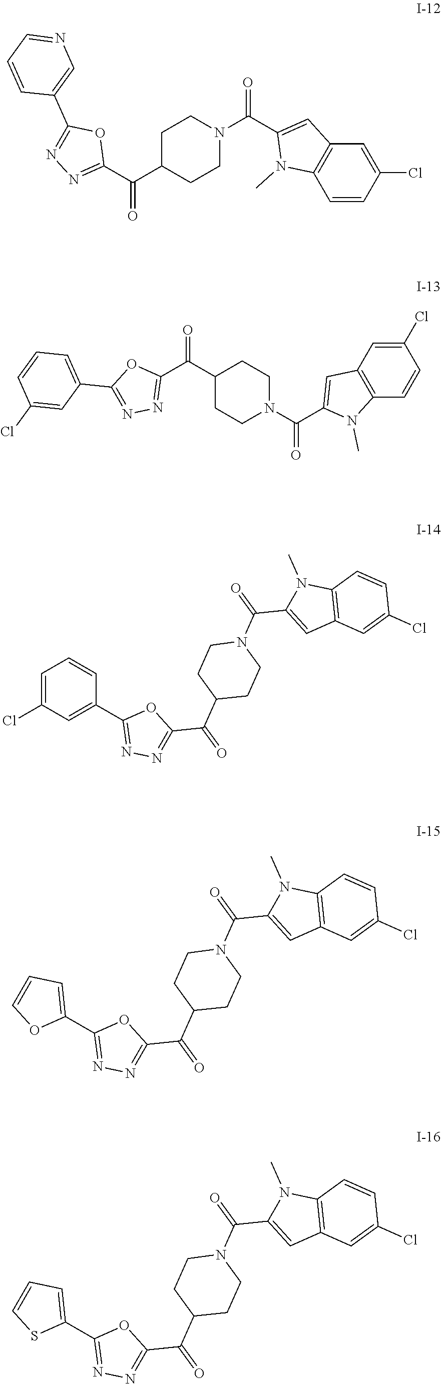 [SMILES: Cn1c(C(=O)N2CCC(C(=O)c3nnc(-c4cccnc4)o3)CC2)cc2cc(Cl)ccc21 ; Cn1c(C(=O)N2CCC(C(=O)c3nnc(-c4cccc(Cl)c4)o3)CC2)cc2cc(Cl)ccc21 ; Cn1c(C(=O)N2CCC(C(=O)c3nnc(-c4cccc(Cl)c4)o3)CC2)cc2cc(Cl)ccc21 ; Cn1c(C(=O)N2CCC(C(=O)c3nnc(-c4ccco4)o3)CC2)cc2cc(Cl)ccc21 ; Cn1c(C(=O)N2CCC(C(=O)c3nnc(-c4cccs4)o3)CC2)cc2cc(Cl)ccc21]

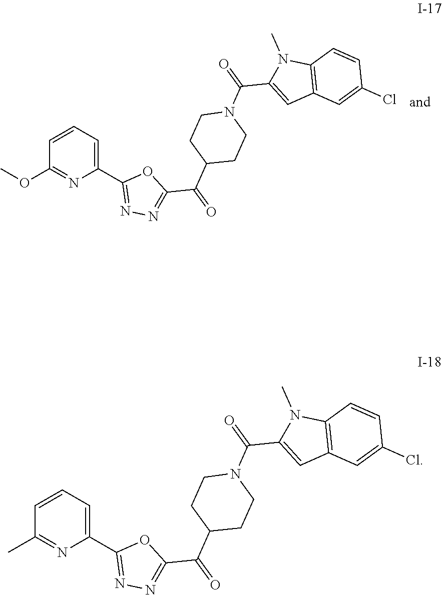 [SMILES: COc1cccc(-c2nnc(C(=O)C3CCN(C(=O)c4cc5cc(Cl)ccc5n4C)CC3)o2)n1 ; "and" ; Cc1cccc(-c2nnc(C(=O)C3CCN(C(=O)c4cc5cc(Cl)ccc5n4C)CC3)o2)n1]

9. A method for preventing and/or treating a FAAH-related disease in a subject in need thereof, comprising administering the compound of formula I or the pharmaceutically acceptable salt thereof according to claim 1.

10. The method according to claim 9, wherein, the FAAH-related disease comprises inflammatory disease, rheumatoid arthritis, hepatitis, hepatic fibrosis, autoimmune disease, pain, depression, pain and depression comorbidity, autism, social anxiety disorder, Tourette's syndrome, neurode-generative disease, anxiety and post-traumatic stress disorder, cannabinoid use disorders, drug withdrawal and anti-tumor treatment.

11. A method for preventing and/or treating a FAAH-related disease in a subject in need thereof, comprising administering the compound of formula I or the pharmaceutically acceptable salt thereof according to claim 8.

12. The method according to claim 11, wherein, the FAAH-related disease comprises inflammatory disease, rheumatoid arthritis, hepatitis, hepatic fibrosis, autoimmune disease, pain, depression, pain and depression comorbidity, autism, social anxiety disorder, Tourette's syndrome, neurode-generative disease, anxiety and post-traumatic stress disorder, cannabinoid use disorders, drug withdrawal and anti-tumor treatment.

13. The compound according to claim 1, wherein,
Ar is selected from pyrrole, oxazole, isoxazole, imidazole, pyrazole, thiazole, isothiazole, triazole, 1,3,4-oxadiazole, 1,3,4-thiadiazole, 1,2,4-oxadiazole, 1,2,4-thiadiazole, benzoxazole, benzothiazole, purine, quinoline, indazole, oxazolo[4,5-b]pyridine, and oxazolo[5,4-b]pyridine.

14. The compound according to claim 1, wherein,
R₁ is H, phenyl, furanyl, thiophenyl, and pyridinyl; the substituent on the phenyl, furanyl, thiophenyl, and pyridinyl is selected from H, F, Cl, Br, I, C₁-C₁₀ straight or branched alkoxy, C₁-C₁₀ straight or branched halogenated alkyl, and C₁-C₁₀ straight or branched alkyl.
